SEQUENCE LISTING (16 sequences)

SEQ ID NO: 1 - protein, 163 aa, synthetic construct
  REGION 1..163: note = MyD88 Domain
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGVDTRRAKR

SEQ ID NO: 2 - protein, 155 aa, synthetic construct
  REGION 1..155: note = MyD88
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLG

SEQ ID NO: 3 - protein, 349 aa, synthetic construct
  REGION 1..349: note = CD3z domain fused to a MyD88 domain
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRMHVDMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGVDTRRAKREGRGSLLTCGDVEENPGP

SEQ ID NO: 4 - protein, 400 aa, synthetic construct
  REGION 1..400: note = CD8a domain linked to a MyD88 domain
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVGGGGSGGGGSMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLG

SEQ ID NO: 5 - DNA, 489 bp, synthetic construct
  misc_feature 1..489: note = MyD88
  source 1..489: mol_type = other DNA
atggcagcaggaggaccaggcgccggcagcgccgcccccgtgagctctaccagctctctgcctttagccgccctgaacatgcgcgtgagaaggcgcctgagcctgtttctgaatgtgcggactcaggtcgccgccgactggacagccctggctgaagaaatggattttgagtacctggagatccgccaattagagacacaggccgatccaacaggccggctgctggatgcctggcagggcagacccggagcctccgtgggcaggctgctggaactgctgactaagctgggcagagatgatgtgctgctggaactgggcccctctatcgaggaggactgtcagaaatacatcctgaagcaacaacaggaagaagcagagaagcctctgcaggtggcagccgtggattctagcgtgccaaggacagccgaactggccggcattaccacactggatgatcctctgggagtcgacacgcgtagagccaagaga

SEQ ID NO: 6 - DNA, 465 bp, synthetic construct
  misc_feature 1..465: note = MyD88
  source 1..465: mol_type = other DNA
atggcagcagggggccctggagctggaagtgccgcaccagtaagtagcacttccagtcttcccctcgcggcactgaatatgagagttcgccggcggttgagcctcttcttgaatgttagaacgcaagtggctgctgactggacagccctcgcggaggaaatggatttcgaatatttggagattaggcaattggagactcaagcagatccgacaggtcgccttcttgacgcttggcagggtcggccgggcgctagtgtgggacgactgcttgagttgctgaccaaattgggccgggacgatgttctcttggagcttggcccatccatagaggaggactgtcaaaaatatatcctcaaacagcagcaggaagaagctgaaaaacctctgcaggtcgcggctgtcgacagcagcgtacctcgaacagctgagttggcgggaataacaacactggatgatccgcttggg

SEQ ID NO: 7 - DNA, 1047 bp, synthetic construct
  misc_feature 1..1047: note = CD3z domain fused to a MyD88 domain
  source 1..1047: mol_type = other DNA
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcatgcatgtcgacatggcagcaggaggaccaggcgccggcagcgccgcccccgtgagctctaccagctctctgcctttagccgccctgaacatgcgcgtgagaaggcgcctgagcctgtttctgaatgtgcggactcaggtcgccgccgactggacagccctggctgaagaaatggattttgagtacctggagatccgccaattagagacacaggccgatccaacaggccggctgctggatgcctggcagggcagacccggagcctccgtgggcaggctgctggaactgctgactaagctgggcagagatgatgtgctgctggaactgggcccctctatcgaggaggactgtcagaaatacatcctgaagcaacaacaggaagaagcagagaagcctctgcaggtggcagccgtggattctagcgtgccaaggacagccgaactggccggcattaccacactggatgatcctctgggagtcgacacgcgtagagccaagagagagggcaggggctctctgctgacatgcggcgatgtggaggagaatcccggccct

SEQ ID NO: 8 - DNA, 1200 bp, synthetic construct
  misc_feature 1..1200: note = CD8a domain linked to a MyD88 domain
  source 1..1200: mol_type = other DNA
atggccttgccggtcacagccctgcttctcccactggcccttctccttcatgcggcgcggccgagtcagttcagagtttcaccccttgacagaacctggaacctcggcgagacagtcgagctgaaatgccaagtgttgcttagcaatccaacttccggatgctcatggttgttccaaccacggggcgccgcagcctcaccaacctttctgttgtacttgtcacagaacaaacctaaggcagcggaaggactcgatacgcaaagattcagcggaaagcgcttgggggacacatttgttttgacgttgtctgatttcagacgcgaaaatgaggggtactatttctgtagtgcgcttagtaacagtattatgtacttttctcattttgtgcctgtattcttgccagcaaagcccacgaccactcctgccccccgacctcccacaccagcgccaaccattgcgtctcaacctcttagcttgcgccctgaagcatgtcggccagcagccggaggtgcggtgcatacaagaggtctcgacttcgcgtgcgatatctacatctgggctcccctggctggaacctgcggggtgctcttgttgtcccttgtcatcacattgtattgcaatcacaggaatagacgaagagtgtgcaagtgtccgcgaccagtggtaaaatcaggtgataaaccaagtcttagcgctcggtacgtgggtggtggcggctctggagggggtgggagcatggcagcagggggccctggagctggaagtgccgcaccagtaagtagcacttccagtcttcccctcgcggcactgaatatgagagttcgccggcggttgagcctcttcttgaatgttagaacgcaagtggctgctgactggacagccctcgcggaggaaatggatttcgaatatttggagattaggcaattggagactcaagcagatccgacaggtcgccttcttgacgcttggcagggtcggccgggcgctagtgtgggacgactgcttgagttgctgaccaaattgggccgggacgatgttctcttggagcttggcccatccatagaggaggactgtcaaaaatatatcctcaaacagcagcaggaagaagctgaaaaacctctgcaggtcgcggctgtcgacagcagcgtacctcgaacagctgagttggcgggaataacaacactggatgatccgcttggg

SEQ ID NO: 9 - protein, 271 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAAAMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLG

SEQ ID NO: 10 - DNA, 813 bp, synthetic construct
  source 1..813: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgcggccgcaatggcagcaggaggaccaggcgccggcagcgccgcccccgtgagctctaccagctctctgcctttagccgccctgaacatgcgcgtgagaaggcgcctgagcctgtttctgaatgtgcggactcaggtcgccgccgactggacagccctggctgaagaaatggattttgagtacctggagatccgccaattagagacacaggccgatccaacaggccggctgctggatgcctggcagggcagacccggagcctccgtgggcaggctgctggaactgctgactaagctgggcagagatgatgtgctgctggaactgggcccctctatcgaggaggactgtcagaaatacatcctgaagcaacaacaggaagaagcagagaagcctctgcaggtggcagccgtggattctagcgtgccaaggacagccgaactggccggcattaccacactggatgatcctctggga

SEQ ID NO: 11 - protein, 891 aa, synthetic construct
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNFGGGKLIFGQGTELSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSLIKVDEFFERAKREGRGSLLTCGDVEENPGPMRIRLLCCVAFSLLWAGPVIAGITQAPTSQILAAGRRMTLRCTQDMRHNAMYWYRQDLGLGLRLIHYSNTAGTTGKGEVPDGYSVSRANTDDFPLTLASAVPSQTSVYFCASSLSFGTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDSRGMHGGGGSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAAAMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLG

SEQ ID NO: 12 - DNA, 2673 bp, synthetic construct
  source 1..2673: mol_type = other DNA
atgaaatccttgagagttttactagtgatcctgtggcttcagttgagctgggtttggagccaacagaaggaggtggagcaaaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgtgaacttcggaggaggaaagcttatcttcggacagggaacggagttatctgtgaaacccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaggtggccgggtttaatctgctcatgacgctgcggctgtggtccagcttaattaaagtcgacgaattcttcgaaagagccaaaagagagggcagaggaagtcttctaacatgcggtgacgtggaggagaatcccggccctatgagaatcaggctcctgtgctgtgtggccttttctctcctgtgggcaggtccagtgattgctgggatcacccaggcaccaacatctcagatcctggcagcaggacggcgcatgacactgagatgtacccaggatatgagacataatgccatgtactggtatagacaagatctaggactggggctaaggctcatccattattcaaatactgcaggtaccactggcaaaggagaagtccctgatggttatagtgtctccagagcaaacacagatgatttccccctcacgttggcgtctgctgtaccctctcagacatctgtgtacttctgtgccagcagcctaagtttcggcactgaagctttctttggacaaggcaccagactcacagttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggattccagaggcatgcatggtggaggtggaagtagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgcggccgcaatggcagcaggaggaccaggcgccggcagcgccgcccccgtgagctctaccagctctctgcctttagccgccctgaacatgcgcgtgagaaggcgcctgagcctgtttctgaatgtgcggactcaggtcgccgccgactggacagccctggctgaagaaatggattttgagtacctggagatccgccaattagagacacaggccgatccaacaggccggctgctggatgcctggcagggcagacccggagcctccgtgggcaggctgctggaactgctgactaagctgggcagagatgatgtgctgctggaactgggcccctctatcgaggaggactgtcagaaatacatcctgaagcaacaacaggaagaagcagagaagcctctgcaggtggcagccgtggattctagcgtgccaaggacagccgaactggccggcattaccacactggatgatcctctggga

SEQ ID NO: 13 - protein, 331 aa, synthetic construct
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRMHVDMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGVDTRRAKR

SEQ ID NO: 14 - DNA, 993 bp, synthetic construct
  source 1..993: mol_type = other DNA
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcatgcatgtcgacatggcagcaggaggaccaggcgccggcagcgccgcccccgtgagctctaccagctctctgcctttagccgccctgaacatgcgcgtgagaaggcgcctgagcctgtttctgaatgtgcggactcaggtcgccgccgactggacagccctggctgaagaaatggattttgagtacctggagatccgccaattagagacacaggccgatccaacaggccggctgctggatgcctggcagggcagacccggagcctccgtgggcaggctgctggaactgctgactaagctgggcagagatgatgtgctgctggaactgggcccctctatcgaggaggactgtcagaaatacatcctgaagcaacaacaggaagaagcagagaagcctctgcaggtggcagccgtggattctagcgtgccaaggacagccgaactggccggcattaccacactggatgatcctctgggagtcgacacgcgtagagccaagaga

SEQ ID NO: 15 - DNA, 993 bp, synthetic construct; identical to SEQ ID NO: 14
  source 1..993: mol_type = other DNA

SEQ ID NO: 16 - protein, 331 aa, synthetic construct; identical to SEQ ID NO: 13

(19) United States
(12) Patent Application Publication
(10) Pub. No.: US 2025/0195573 A1
(43) Pub. Date: Jun. 19, 2025

(54) GENETICALLY ENGINEERED T-CELL CO-RECEPTORS AND METHODS OF USE THEREOF

(71) Applicant: The Regents of the University of Colorado, A Body Corporate, Denver, CO (US)

(72) Inventor: Eduardo DAVILA, Centennial, CO (US)

(21) Appl. No.: 18/847,963

(22) PCT Filed: Mar. 20, 2023
(86) PCT No.: PCT/US2023/064708

§ 371 Date: Sep. 17, 2024

Related U.S. Application Data
(60) Provisional application No. 63/321,553, filed on Mar. 18, 2022.

(51) Int. Cl. A61K 35/17; A61K 40/11; A61K 40/32; A61K 40/42; A61P 35/00; C07K 14/725; C12N 5/0783

(52) U.S. Cl. CPC A61K 35/17; A61K 40/11; A61K 40/32; A61K 40/4272; A61P 35/00; C07K 14/7051; C12N 5/0636; A61K 2239/28; A61K 2239/49; A61K 2239/55; A61K 2239/57; C12N 2510/00

(57) ABSTRACT

This disclosure relates to modified receptors comprising a MyD88 domain, as well as cells comprising the same and methods of use thereof.

### CROSS-REFERENCE TO RELATED APPLICATIONS

The present application claims the benefit of U.S. Provisional Patent Application No. 63/321,553, filed Mar. 18, 2022, which is incorporated herein by reference in its entirety.

### STATEMENT REGARDING FEDERALLY SPONSORED RESEARCH

This invention was made with government support under R01CA207913 awarded by the National Institute of Health. The government has certain rights in the invention.

### INCORPORATION-BY-REFERENCE OF SEQUENCE LISTING

The Sequence Listing XML associated with this application is provided electronically in XML file format and is hereby incorporated by reference into the specification. The name of the XML file containing the Sequence Listing XML is “UNCO-040_SeqList_ST26”. The XML file is 29,452 bytes, created on Mar. 20, 2023, and is being submitted electronically via USPTO Patent Center.

### FIELD

This disclosure relates to modified receptors and fusion proteins comprising a MyD88 domain and a CD3 subunit, as well as T cell receptors and cells comprising the same and methods of use thereof.

### BACKGROUND

Adoptive cell therapies, including therapy with TILs, and CAR T cells, as well as TCR therapy, have shown promise in the treatment of hematologic and solid tumors. However, CAR-T cell therapies are currently the only FDA approved T-cell therapies (for example, Yescarta, Kymriah, Tecartus, Abecma, and Breyanzi).

T cell therapies have several limitations that have hindered clinical development. For example, cancer patients undergoing treatment with cellular therapies such as CAR T cells. Natural-Killer (NK) cells. tumor-infiltrating lymphocytes (TIL), or T-cell receptor (TCR) engineered cells must undergo a preconditioning step using chemotherapies to promote the engraftment of immune cells. Patients receiving TILs also receive high-dose IL-2. Unfortunately, the preconditioning step leads to transient depletion of not only the endogenous T cells but also other important immune cells, platelets, and red blood cells. High-dose IL-2 is highly toxic and requires in-patient service in intensive care units due to high risk of developing toxicities.

In addition to the toxic effects on the patient, lymphodepletion and IL-2 are expensive, and differentiated cells lose efficacy in vivo quickly, limiting their therapeutic efficacy.

There thus remains an unmet need for cellular therapies that do not require lymphodepletion and/or IL-2 therapy for the engraftment and expansion of T cells

### SUMMARY

In one aspect, provided herein is a cell comprising (a) a modified T cell receptor (TCR) comprising an α chain, a β chain, a CD3ζ, intracellular domain and a MyD88 polypeptide comprising the death domain (DD) of MyD88 and the intermediate domain (ID) of MyD88, wherein the MyD88 domain is fused to the CD3ζ, subunit, or (b) a fusion protein comprising a CD3ζ, intracellular domain and a MyD88 DD. In some embodiments, the MyD88 polypeptide comprises an amino acid sequence that is at least 90% identical to the sequence set forth in SEQ ID NO: 1 or 2. In some embodiments, the fusion protein comprises an amino acid sequence that is at least 90% identical to the amino acid sequence set forth in SEQ ID NO: 3 or 9.

In another aspect, provided herein is a cell comprising (a) a modified T cell receptor (TCR) comprising an α chain, a 0 chain, a CD3ζ intracellular domain and a MyD88 polypeptide comprising an amino acid sequence that is at least 90% identical to the sequence set forth in SEQ ID NO: 1 or 2, wherein the MyD88 domain is fused to the CD3ζ subunit, or (b) a fusion protein comprising an amino acid sequence that is at least 90% identical to the amino acid sequence set forth in SEQ ID NO: 3 or 9.

In another aspect, provided herein is a modified T cell receptor (TCR) comprising an α chain, a β chain, and the CD3 subunits CD3γ, CD3ζ, CD3ε, and CD3δ, wherein a MyD88 domain comprising the death domain (DD) of MyD88 and the intermediate domain (ID) of MyD88 is fused to the CD3ζ subunit. In some embodiments, the T cell receptor targets a cancer antigen. In some embodiments, the MyD88 domain comprises the sequence set forth in SEQ ID NO: 1 or 2.

In another aspect, provided herein is a cell comprising a modified TCR described herein.

In another aspect, provided herein is a pharmaceutical composition comprising a cell described herein and a pharmaceutically acceptable carrier.

In another aspect, provided herein is a method of treating cancer in a subject in need thereof, the method comprising administering to the subject a cell or a pharmaceutical composition described herein. In some embodiments, the patient does not undergo lymphodepletion prior to administration of the T cell. In some embodiments, the patient does not receive cytokines while being administered the cell. In some embodiments, the cancer is breast cancer, sarcoma, melanoma, or lung cancer.

In another aspect, provided herein is a method of improving a therapeutic cell, the method comprising transfecting the therapeutic cell with a modified TCR described herein.

In another aspect, provided herein is a method of improving a therapeutic cell, the method comprising modifying a receptor of the therapeutic cell to fuse a MyD88 domain to a CD3ζ domain present in the receptor of the therapeutic cell. In some embodiments, the MyD88 domain comprises the sequence set forth in SEQ ID NO: 1 or 2.

In another aspect, provided herein is a method of improving a therapeutic cell, the method comprising introducing into the cell a fusion protein comprising a CD3ζ intracellular domain and a MyD88 DD. In some embodiments, the fusion protein comprises an amino acid sequence that is at least 90% identical to the amino acid sequence set forth in SEQ ID NO: 3 or 9. In some embodiments, the cell is an immune cell. In some embodiments, the cell is derived from a patient.

In some embodiments, the method results in an increase in expansion of the therapeutic cell in vivo of at least 50% compared to the therapeutic cell prior to improvement.

In some embodiments, the method results in an increase in cytokine expression of the therapeutic cell in vivo of at least 50% compared to the therapeutic cell prior to improvement.

In some embodiments, the method results in a decrease in exhaustion of the therapeutic cell in vivo of at least 20% compared to the therapeutic cell prior to improvement. In some embodiments, the therapeutic cell retains comparable antigen specificity after transfection.

### DESCRIPTION OF DRAWINGS

FIGS. 1A and 1B show four T cell receptor (TCR) constructs comprising a α and β TCR of the TCR known as “DMF5” that is reactive towards the MART-1 (amino acids 27-35). A CD3z:MyD88was added to the α and/or p TCR chain. Only the intracellular (ICS) domain of the CD3z was used. The MyD88 lacked the toll/interleukin-1 (IL-1) receptor (TIR). FIG. 1A shows a schematic of DNA constructs which may be cloned into a vector (e.g., a pMSGV retroviral vector). FIG. 1B shows a schematic of TCR constructs which may be expressed on the T cell surface. LS, human CD8alpha leader sequence; α chain/β chain, human α/β chains of DMF5 TCR; G4S, GGGGS Linker Sequence; CD3z_ICS, intracellular domain of CD3z, MyD88=Death and Intermediate domains of MyD88, EGFRt, transmembrane Endothelial growth factor receptor. The EGFRt is used as a reporter to determine transduction efficiency. It can also be used as a means to sort transduced cells using reagents (ie antiEGFR antibodies). Alternatively, EGFRt can also be used to target cells for tracking in vivo using labeled antiEGFR antibodies. EGFRt can also be used as a target to destroy transduced T cells using an antiEGFR antibody drug conjugate. FIG. 1C shows the various proteins that make up the T cell receptor complex, including the T cell receptor alpha and beta chains, and the CD3 subunits, CD3γ, CD3ζ, CD3ε, and CD3δ. CD3ζ forms dimers with CD3γ and CD3δ, while CD3ζ forms homodimers. Novel synthetic CD3 signaling molecules were developed by engineering CD3ε (“ε-amdTCR”) and CD3ζ (ζ-ampTCR) to express the MyD88 death and intermediate domain. FIG. 1D shows the CD3ζ:MyD88 fusion protein without the TCR complex. This rendition offers a better view of the dimerized CD3 ζ:MyD88 fusion protein in the absence of other TCR signaling molecules.

FIG. 2 shows expression of TCR:CD3z:MyD88 constructs in human T cells as determined by flow cytometry.

FIGS. 3A and 3B show effector cytokine expression in T cells engineered with the DMF5 CD3z:MyD88 appended to the TCRbeta or TCRalpha chain. FIG. 3A shows cytokine expression in Jurkat cells engineered to express the DMF5 TCR:CD3c:MyD88 constructs. Each point represents the mean of three technical replicates, error bars=1 standard deviation. *indicates significant difference (p<0.05) between DMF5 α:CD3z:MyD88 and DMF5 β:CD3z:MyD88 groups with Wild Type DMF5 but not with each other and **indicates significant difference (p<0.05) between α:CD3z:MyD88, β:CD3z:MyD88 groups, Wild Type each with each other by one-way Anova. FIG. 3B shows cytokine expression in 624 melanoma cells (naturally expressing HLA-A2+MART1+). Each point represents one technical replicate, each bar represents mean of replicates, error bars=1 standard deviation. p-values are as indicated in the graphs and were determined by one-way ANOVA.

FIGS. 4A and 4B show response and polyfunctionality of T cells engineered with the TCRB:CD3z:MyD88, wild type TCR, or control TCR:CD3z. FIG. 4A shows expression of TNFα and TNFβ. FIG. 4B shows expression of IL-2 and GM-CSF.

FIGS. 5A and 5B show cytokine production in T cells engineered with the TCRB:CD3z:MyD88 compared to cells engineered with wild type TCR, TCRA:CD3z:MyD88, or TCR:CD3z. FIG. 5A shows a comparisons between DMF5 WT, TCRB:CD3z, and TCRB:CD3z:MyD88. FIG. 5B shows a comparison between DMF5 WT, TCRB:CD3z, TCRA:CD3z:MyD88, and TCRB:CD3z:MyD88. Each dot represents the mean of three technical replicates, error bars=1 Standard Deviation. FIG. 5A includes IL-2 production. FIG. 5B shows data using a TCR in which MyD88 has been appended to both the TCR alpha and TCR beta. Withou wishing to be bound by theory, appending MyD88 to both TCR chains is believed to prevent the TCR from being expressed and becomes a non-functional receptor.

FIGS. 6A and 6B show the effects of TCRB:CD3z:MyD88 on T cell expansion. FIG. 6A shows a schematic of the experimental set up. FIG. 6B shows cells counts for T cells from three donors engineered with DMF5 TCR or TCRB:CD3Z:MyD88.

FIG. 7 shows the effect of TCRB:CD3z:MyD88 on T cells anti-tumor activity and effect molecule production.

FIGS. 8A-8C show the effects of CD3z and CD3z-MyD88 on intracellular levels of cytokines as determined by flow cytometry (FIG. 8A), as well as levels of polyfunctional TILs (those that produce more than one cytokine, FIGS. 8B and 8C).

FIGS. 9A-9F show proliferation of unmodified TILs and TILs transfected with control CD3z or CD3z:MyD88 in vivo in breast cancer cells (FIG.s. 9A and 9C), sarcoma cells (FIGS. 9B and 9D), and melanoma cells (FIGS. 9E and 9F).

FIG. 10 shows the TCR diversity and repertoire is in unmodified TILs and TILs transfected with control CD3z or CD3z:MyD88. Each population represents one TCRVs family member.

FIG. 11A and 11B show polyfunctionality of unmodified TILs and TILs transfected with control CD3z or CD3z:MyD88 over time. FIG. 1I A shows intracellular levels of cytokines on T cells stimulated with anti-CD3 antibody. FIG. 11B shows the percentage of T cells capable of co-producing various effector milecules at different time points.

FIGS. 12A-12D show the effect of CD3z:MyD88 on T cell activity (based on IFNγ production). Shown are data in breast cancer (FIG. 12A), sarcoma (FIG. 12B) and melanoma (FIGS. 12C and 12D) samples.

FIGS. 13A and 13B show proliferaftion advantages of T cells transfected with CD3ζ:MyD88. FIG. 13A shows the proliferation fo CD4+ and CD8+ T cells with and without simulation by anti-CD3 antibody. FIG. 13B shows mean fluorescence intensity of a proliferation dye in CD8+ T cells.

FIGS. 14A-14C show cytokine production in CD3z:MyD88 cells. FIG. 14A shows IFNγ production assessed by ELISA in T cells stimulated with varying concentrations of the gp100 antigen or with B16 tumor cells. FIG. 14B shows production of TNFα and IFNγ as determined by intracellular FACS. FIG. 14C summarizes the numbers of cytokines expressed by each cell population.

FIGS. 15A-15C shows the antitumor effect of TILs transfected with control CD3z or CD3z:MyD88 One of 3 representative experiments shown (n=4-5 mice per group). FIGS. 15A and 15B show tumor size of B16 melanomas in mice after preconditioning with radiation followed by treatment with PBS or T cells expressing the control CD3z construct or T cells expressing the CD3z:MyD88 construct, with FIG. 15A showing data for each animal in the group for one of 3 representative experiments shown (n=4-5 mice per group), and FIG. 15B showing the average for each group. FIG. 15C shows survival of mice carrying B16 melanomas after treatment with radiation and PBS or T cells expressing the control CD3z construct or T cells expressing the CD3z:MvD88 construct. Statistical significance was determined by the mixed effect ANOVA model with Geisser Greenhouse correction and Tukey's multiple comparisons test *** p<0.001. Error bars represent the standard mean deviation from the mean of 5 mice per group.

FIGS. 16A-16C show body weight of animals treated with PBS, or with T cells expressing the control CD3z construct or with T cells expressing CD3z:MyD88. FIGS. 16A-16C each show data for one of three experiments. The x-axes of FIGS. 16A-16C show days.

FIGS. 17A-17C show the effect of CD3z:MyD88 on neoantigen-reactive TILs. FIG. 17A shows a schematic of the experimental set up. FIG. 17B shows INFγ levels in TILs engineered to express CD3z:MyD88 (grey bars) as compared with CD3z (black bars). FIG. 17C shows the number of INFγ producing cells and usage of TCRvB family genes as determined by flow cytometry.

### DETAILED DESCRIPTION

Provided herein are synthetic T cell receptors (TCRs) which comprise a MyD88 domain. Also provided herein are cells comprising the TCRs described herein and methods of using the same.

### Modified Receptors

Modified T cell receptors can be used in adoptive T cell therapy to harness the body's immune system to to targets, e.g., cancer cell. In the context of chimeric antigen receptors (CARs), an immune cell is transfected to express an artificial receptor which binds to a predetermined antigen. TCR therapy relies on the ability of the TcR to recognize antigens presented by major histocompatibility complex (MHC) molecules. See, e.g., Zhao and Cao, 2019; Front. Immunol. 10:2250, which is incorporated herein by reference in its entirety.

The T cell receptor complex is composed of various proteins, including the T cell receptor alpha and beta chains, and the CD3 subunits, CD3γ, CD3ζ, CD3ε, and CD3δ. CD3ε forms dimers with CD3γ and CD3δ, while CD3ζ forms homodimers. The α chain and the β chain each comprise a constant region and a variable region. The variable regions of the α chain and the β chain confers antigen specificity of the receptor. Thus, there are three complementarity-determining regions (CDRs) in each of the α chain and the δ chain, with the CDR1 and CDR2 typically contacting the MHC molecule, while the CDR3 usually contacts the antigen. See Wong et al., 2019, Front Immuno. 10:2454, which is incorporated herein by reference in its entirety. A TCR is activated upon binding to an antigen presented by MHC.

A CAR generally comprises an extracellular domain comprising an antigen-binding region (e.g., an antibody or a part of an antibody such as a single chain variable fragment (scFv)), a transmembrane domain, and an intracellular domain. The intracellular domain often comprises a CD3ζ signaling domain and one or two costimulatory domains, which may be derived from, for example, CD8, CD28, OX40 or 4-1BB, or be a combination of the same. See Jayaraman et al., EBioMedicine 58 (2020) 102931, which is incorporated herein by reference in its entirety.

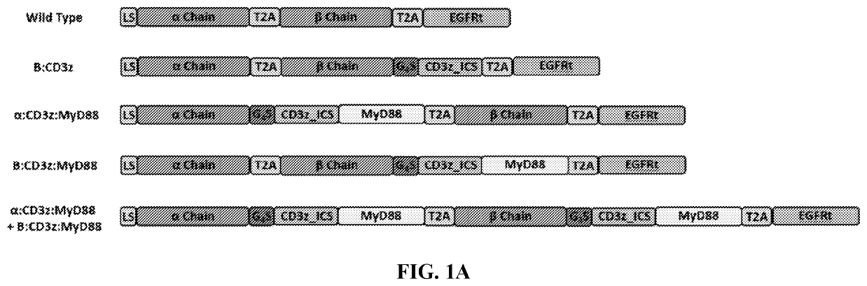

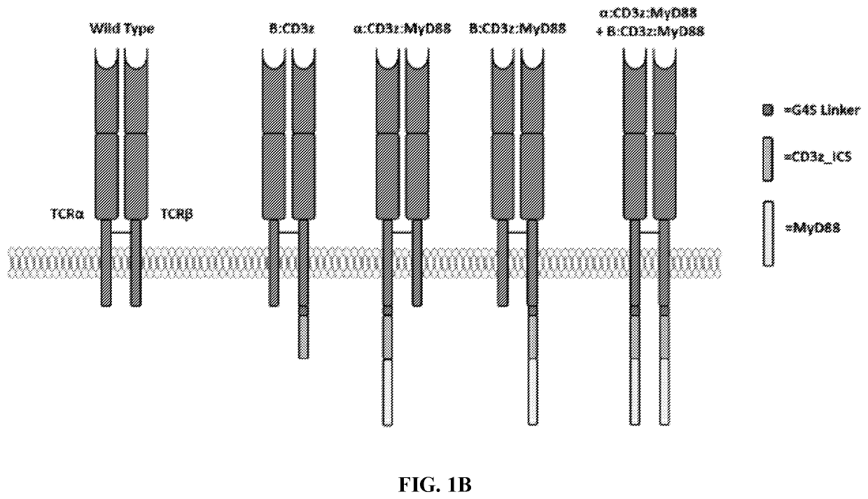

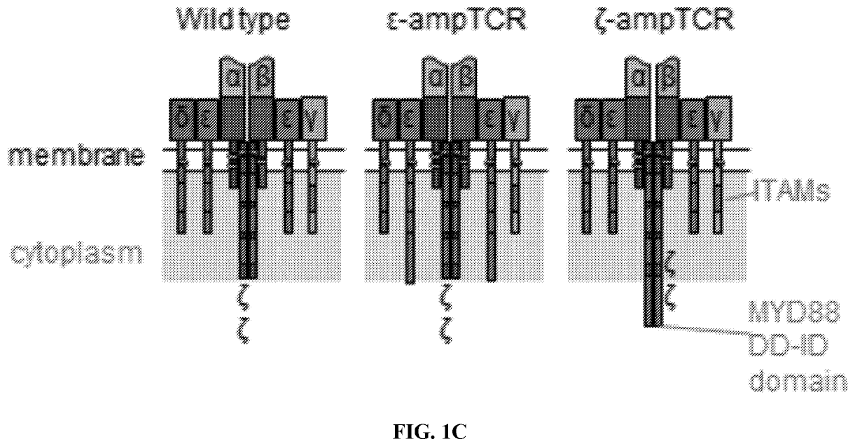

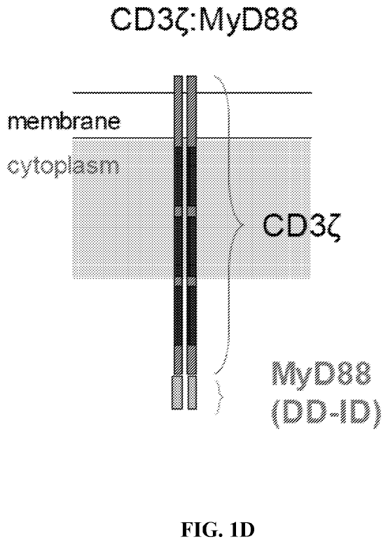

In some embodiments, the CD3ζ domain is linked or fused to the α chain of a TCR In some embodiments, the CD3ζ domain is linked or fused to the β chain of a CDR In some embodiments, one CD3ζ subunit is linked to each of the α chain and the β chain of the TCR. Schematics of constructs illustrating these four arrangement are shown in FIGS. 1A-1C.

Provided herein are modified TCRs comprising an α chain, a β chain, and the CD3 subunits CD3γ, CD3ζ, CD3ε, and CD3δ, wherein the CD3ζ or the CD3ε subunit is fused to a MyD88 domain. MyD88 is an adaptor for signaling pathways including the Toll-like receptor (TLR) and interleukin-1 (IL-1) receptor families. MyD88 comprises three main domains: an N-terminal death domain (DD), an intermediate domain (INT), and a C-terminal Toll-interleukin-1 receptor domain (TIR). MyD88 usually induces pro-inflammatory signaling, but depending on the contexts can have anti-inflammatory effects. The DD of MyD88 associates with the IRAK family members and overexpression of the DD leads to NFκB activation and c-Jun N-terminal kinase (JNK) activation. The TIR domain mediates the interaction with other TIR domain-containing proteins. The INT domain links the TIR and the DD, and variants of MyD88 lacking the INT domain acts as a dominant negative form of MyD88. See Deguine and Barton, F1000Prime Reports 2014, 6:97, which is incorporated herein by reference in its entirety.

In some embodiments, a modified receptor provided herein is a modified TCR comprising a MyD88 domain linked or fused to the CD3ζ or the CD3ε subunit. In some embodiments, a modified receptor provided herein is a CAR comprising a MyD88 domain linked or fused to the CD3ζ domain.

In some embodiments, the MyD88 domain comprises the DD and/or the INT of MyD88.

Illustrative sequences of MyD88 domains comprising the DD and the INT are set forth in SEQ ID NOs: 1 and 2:

(SEQ ID NO: 1)
MAAGGPGAGSAAPVSSTSSLPLAALNMRVRRRLSLFLNVRTQVAADWTAL
AEEMDFEYLEIRQLETQADPTGRLLDAWQGRPGASVGRLLELLTKLGRDD
VLLELGPSIEEDCQKYILKQQQEEAEKPLQVAAVDSSVPRTAELAGITTL
DDPLGVDTRRAKR.
(SEQ ID NO: 2)
MAAGGPGAGSAAPVSSTSSLPLAALNMRVRRRLSLFLNVRTQVAADWTAL
AEEMDFEYLEIRQLETQADPTGRLLDAWQGRPGASVGRLLELLTKLGRDD
VLLELGPSIEEDCQKYILKQQQEEAEKPLQVAAVDSSVPRTAELAGITTL
DDPLG.

In some embodiments, a modified receptor provided herein comprises a sequence that is at least 80%, at least 85%, at least 90%, at least 91%, at least 92%, at least 93%, at least 94%, at least 95%, at least 96%, at least 97%, at least 98%, or at least 99% identical to the sequence set forth in SEQ ID NO: 1. In some embodiments, a modified receptor provided herein comprises the amino acid sequence set forth in SEQ ID NO: 1 with one, two, three, four or five conservative amino acid substitutions. In some embodiments, a modified receptor provided herein comprises the sequence set forth in SEQ ID NO: 1.

A “conservative amino acid substitutions” is an amino acid substitution that replaces one amino acid with another amino acid which has similar properties. For example, a hydrophobic amino acid (Ala, Cys, Gly, Pro, Met, Val, Ile, Leu) is substituted with another hydrophobic amino acids, a hydrophobic amino acid with a bulky side chain (Phe, Tyr, Trp) is substituted with other hydrophobic amino acid with a bulky side chain, an amino acid with a positively charged side chain (Arg, His, Lys) is substituted with another amino acid with a positively charged side chain, an amino acid with a negatively charged side chain (Asp, Glu) is substituted with another amino acids with a negatively charged side chain, or an amino acid with a polar uncharged side chain (Ser, Thr, Asn, Gln) is substituted with another amino acid with a polar uncharged side chain.

In some embodiments, a modified receptor provided herein comprises a sequence that is at least 80%, at least 85%, at least 90%, at least 91%, at least 92%, at least 93%, at least 94%, at least 95%, at least 96%, at least 97%, at least 98%, or at least 99% identical to the sequence set forth in SEQ ID NO: 2. In some embodiments, a modified receptor provided herein comprises the amino acid sequence set forth in SEQ ID NO: 2 with one, two, three, four or five conservative amino acid substitutions. In some embodiments, a modified receptor provided herein comprises the sequence set forth in SEQ ID NO: 2.

An illustrative sequence of a CD3ζ domain fused to a MyD88 domain comprising the DD and INT domain is set forth in SEQ ID NO: 3, with the CD3ζ domain underlined, and the MyD88 domain shown in bold. Shown in italics is a T2A self-cleaving fragment that may be included to facilitate transfection and expression of the modified TCR

(SEQ ID NO: 3)
MKWKALFTAAILQAQLPITEAQSFGLLDPKLCYLLDGILFIYGVILTALF
LRVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTATK
DTYDALHMQALPPRMHVDMAAGGPGAGSAAPVSSTSSLPLAALNMRVRRR
LSLFLNVRTQVAADWTALAEEMDFEYLEIRQLETQADPTGRLLDAWQGRP
GASVGRLLELLTKLGRDDVLLELGPSIEEDCQKYILKQQQEEAEKPLQVA
AVDSSVPRTAELAGITTLDDPLGVDTRRAKREGRGSLLTCGDVEENPGP.

In some embodiments, a modified TCR provided herein comprises an amino acid sequence that is at least 75%, at least 80%, at least 85%, at least 90%, at least 91%, at least 92%, at least 93%, at least 94%, at least 95%, at least 96%, at least 97%, at least 98%, or at least 99% identical to amino acids 175-338 of SEQ ID NO; 3. In some embodiments, a modified TCR provided herein comprises amino acids 175-338 of SEQ ID NO; 3. In some embodiments, a modified TCR provided herein comprises an amino acid sequence that is at least 75%, at least 80%, at least 85%, at least 90%, at least 91%, at least 92%, at least 93%, at least 94%, at least 95%, at least 96%, at least 97%, at least 98%, or at least 99% identical to amino acids 1-338 of SEQ ID NO; 3. In some embodiments, a modified TCR provided herein comprises amino acids 1-338 of SEQ ID NO: 3.

An alternative sequence of a CD3ζ domain fused to myD88 domain is shown in SEQ ID NO: 9, with the CD3ζ domain underlined, and the MyD88 domain shown in bold. Shown in italics is a linker sequence.

(SEQ ID NO: 9)
RVKFSRSADAPAYQQGQNQLYNELNLGRREEYDVLDKRRGRDPEMGGKPQ
RRKNPQEGLYNELQKDKMAEAYSEIGMKGERRRGKGHDGLYQGLSTATKD
TYDALHMQALPPRAAAMAAGGPGAGSAAPVSSTSSLPLAALNMRVRRRLS
LFLNVRTQVAADWTALAEEMDFEYLEIRQLETQADPTGRLLDAWQGRPGA
SVGRLLELLTKLGRDDVLLELGPSIEEDCQKYILKQQQEEAEKPLQVAAV
DSSVPRTAELAGITTLDDPLG.

In some embodiments, a modified TCR provided herein comprises an amino acid sequence that is at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 9. In some embodiments, a modified TCR provided herein comprises the amino acid sequence set forth in SEQ ID NO: 9.

Another alternative sequence of a CD3ζ domain fused to myD88 domain is shown in SEQ ID NO: 16, with the CD3ζ domain underlined, and the MyD88 domain shown in bold.

(SEQ ID NO: 16)
MKWKALFTAAILQAQLPITEAQSFGLLDPKLCYLLDGILFIYGVILTALF
LRVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTATK
DTYDALHMQALPPRMHVDMAAGGPGAGSAAPVSSTSSLPLAALNMRVRRR
LSLFLNVRTQVAADWTALAEEMDFEYLEIRQLETQADPTGRLLDAWQGRP
GASVGRLLELLTKLGRDDVLLELGPSIEEDCQKYILKQQQEEAEKPLQVA
AVDSSVPRTAELAGITTLDDPLGVDTRRAKR.

In some embodiments, a modified TCR provided herein comprises an amino acid sequence that is at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 16. In some embodiments, a modified TCR provided herein comprises the amino acid sequence set forth in SEQ ID NO: 16.

It will be apparent to a person of skill in the art that, as an alternative to fusing the MyD88 domain to the CD3 domain (e.g., the CD3ζ domain), the MyD88 domain may be linked to the CD3 domain (e.g., the CD3ζ domain) via a linker. Suitable linkers are known in the art and include, for example, a G4S linker, or GGGS linker.In another aspect, the MyD88 domain may be attached to a cell surface antigen of a T cell. In some embodiments, the MyD88 domain is attached to CD8a. CD8a is a membrane glycoprotein expressed on a subset of T cells which acts as a coreceptor for MHC class I.

An exemplary sequence of a CD8a domain linked to a MyD88 domain comprising the DD and INT domain is set forth in SEQ ID NO: 4, with the CD8a domain underlined, the MyD88 domain shown in bold, and a G4S linker connecting the MyD88 domain and the CD8a domain shown in italics.

(SEQ ID NO: 4)
MALPVTALLLPLALLLHAARPSQFRVSPLDRTWNLGETVELKCQVLLSN
PTSGCSWLFQPRGAAASPTFLLYLSQNKPKAAEGLDTQRFSGKRLGDTF
VLTLSDFRRENEGYYFCSALSNSIMYFSHFVPVFLPAKPTTTPAPRPPT
PAPTIASQPLSLRPEACRPAAGGAVHTRGLDFACDIYIWAPLAGTCGVL
LLSLVITLYCNHRNRRRVCKCPRPVVKSGDKPSLSARYVGGGGSGGGGS
MAAGGPGAGSAAPVSSTSSLPLAALNMRVRRRLSLFLNVRTQVAADWTA
LAEEMDFEYLEIRQLETQADPTGRLLDAWQGRPGASVGRLLELLTKLGR
DDVLLELGPSIEEDCQKYILKQQQEEAEKPLQVAAVDSSVPRTAELAGI
TTLDDPLG.

In some embodiments, a modified TCR provided herein comprises an amino acid sequence that is at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 4. In some embodiments, a modified TCR provided herein comprises the amino acid sequence set forth in SEQ ID NO: 4.

An illustrative amino acid sequence of a modified TCR comprising a TCR α chain, a TCR β chain, a CD3ζ intracellular domain (ICD), and a MyD88 DD is set forth in SEQ ID NO: 11, with the TCR α chain underlined, the TCR β chain shown in bold, the CD3ζ ICD shown in underlined and bold, and the MyD88 DD shown in bold italics. Shown in italics are linker sequences.

(SEQ ID NO: 11)
MKSLRVLLVILWLQLSWVWSQQKEVEQNSGPLSVPEGAIASLNCTYSDRG
SQSFFWYRQYSGKSPELIMFIYSNGDKEDGRFTAQLNKASQYVSLLIRDS
QPSDSATYLCAVNFGGGKLIFGQGTELSVKPNIQNPDPAVYQLRDSKSSD
KSVCLFTDFDSQTNVSQSKDSDVYITDKTVLDMRSMDFKSNSAVAWSNKS
DFACANAFNNSIIPEDTFFPSPESSCDVKLVEKSFETDTNLNFQNLSVIG
FRILLLKVAGFNLLMTLRLWSSLIKVDEFFERAKREGRGSLLTCGDVEEN
PGPMRIRLLCCVAFSLLWAGPVIAGITQAPTSQILAAGRRMTLRCTQDMR
HNAMYWYRQDLGLGLRLIHYSNTAGTTGKGEVPDGYSVSRANTDDFPLTL
ASAVPSQTSVYFCASSLSFGTEAFFGQGTRLTVVEDLNKVFPPEVAVFEP
SEAEISHTQKATLVCLATGFFPDHVELSWWVNGKEVHSGVSTDPQPLKEQ
PALNDSRYCLSSRLRVSATFWQNPRNHFRCQVQFYGLSENDEWTQDRAKP
VTQIVSAEAWGRADCGFTSVSYQQGVLSATILYEILLGKATLYAVLVSAL
VLMAMVKRKDSRGMHGGGGSRVKFSRSADAPAYQQGQNQLYNELNLGRRE
EYDVLDKRRGRDPEMGGKPQRRKNPQEGLYNELQKDKMAEAYSEIGMKGE
RRRGKGHDGLYQGLSTATKDTYDALHMQALPPRAAAMAAGGPGAGSAAPV
SSTSSLPLAALNMRVRRRLSLFLNVRTQVAADWTALAEEMDFEYLEIRQL
ETQADPTGRLLDAWQGRPGASVGRLLELLTKLGRDDVLLELGPSIEEDCQ
KYILKQQQEEAEKPLQVAAVDSSVPRTAELAGITTLDDPLG.

In some embodiments, a modified TCR provided herein comprises an amino acid sequence that is at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 11. In some embodiments, a modified TCR provided herein comprises the amino acid sequence set forth in SEQ ID NO: 11. The modified TCRs described herein may be screened for the ability to bind specific antigens before the modifications are introduced.

Without wishing to be bound by theory, it is believed that the addition of a MyD88 domain to a modified receptor leads to antigen-specific activation of the receptor, and improves the clinical utility of the receptor an cell comprising the same.

### MyD88 Fusion Proteins

Also provided herein are fusion proteins comprising MyD88 domains. These fusion proteins may be used to enhance the endogenous cell-killing abilities of immune cells, including natural killer (NK) cells, T cells, macrophages, and tumor infiltrating lymphocytes (TILS).

An illustrative sequence of a fusion protein comprising a CD3ζ intracellular domain (ICD), and a MyD88 DD is set forth in SEQ ID NO: 13, with the CD3ζ domain underlined, and the MyD88 domain shown in bold.

(SEQ ID NO: 13)
MKWKALFTAAILQAQLPITEAQSFGLLDPKLCYLLDGILFIYGVILTALF
LRVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTATK
DTYDALHMQALPPRMHVDMAAGGPGAGSAAPVSSTSSLPLAALNMRVRRR
LSLFLNVRTQVAADWTALAEEMDFEYLEIRQLETQADPTGRLLDAWQGRP
GASVGRLLELLTKLGRDDVLLELGPSIEEDCQKYILKQQQEEAEKPLQVA
AVDSSVPRTAELAGITTLDDPLGVDTRRAKR.

In some embodiments, a fusion protein provided herein comprises an amino acid sequence that is at least 75%, at least 80%, at least 85%, at least 90%, at least 91%, at least 92%, at least 93%, at least 94%, at least 95%, at least 96%, at least 97%, at least 98%, or at least 99% identical to the amino acid sequence set forth in SEQ ID NO: 13. In some embodiments, a fusion protein provided herein comprises the amino acid sequence set forth in SEQ ID NO: 13.

Without wishing to be bound by theory, it is believed that once a CD3ζ-MyD88 fusion protein has been introduced into a cell, it localizes in the cells membrance and provides stimulation to a cell into which it is introduced, resulting in increased cell proliferation, cytokine secretion and/or anti-tumor activity.

### Polynucleotides and Vectors

Also provided herein are polynucleotides and vectors encoding the modified receptors and the fusion proteins described herein or parts thereof.

Exemplary polynucleotides comprising nucleic acid sequences encoding a MyD88 domain comprising a DD and an INT domain are set forth in SEQ ID NO: 5 and 6.

(SEQ ID NO: 5)
ATGGCAGCAGGAGGACCAGGCGCCGGCAGCGCCGCCCCCGTGAGCTCTAC
CAGCTCTCTGCCTTTAGCCGCCCTGAACATGCGCGTGAGAAGGCGCCTGA
GCCTGTTTCTGAATGTGCGGACTCAGGTCGCCGCCGACTGGACAGCCCTG
GCTGAAGAAATGGATTTTGAGTACCTGGAGATCCGCCAATTAGAGACACA
GGCCGATCCAACAGGCCGGCTGCTGGATGCCTGGCAGGGCAGACCCGGAG
CCTCCGTGGGCAGGCTGCTGGAACTGCTGACTAAGCTGGGCAGAGATGAT
GTGCTGCTGGAACTGGGCCCCTCTATCGAGGAGGACTGTCAGAAATACAT
CCTGAAGCAACAACAGGAAGAAGCAGAGAAGCCTCTGCAGGTGGCAGCCG
TGGATTCTAGCGTGCCAAGGACAGCCGAACTGGCCGGCATTACCACACTG
GATGATCCTCTGGGAGTCGACACGCGTAGAGCCAAGAGA
(SEQ ID NO: 6)
ATGGCAGCAGGGGGCCCTGGAGCTGGAAGTGCCGCACCAGTAAGTAGCAC
TTCCAGTCTTCCCCTCGCGGCACTGAATATGAGAGTTCGCCGGCGGTTGA
GCCTCTTCTTGAATGTTAGAACGCAAGTGGCTGCTGACTGGACAGCCCTC
GCGGAGGAAATGGATTTCGAATATTTGGAGATTAGGCAATTGGAGACTCA
AGCAGATCCGACAGGTCGCCTTCTTGACGCTTGGCAGGGTCGGCCGGGCG
CTAGTGTGGGACGACTGCTTGAGTTGCTGACCAAATTGGGCCGGGACGAT
GTTCTCTTGGAGCTTGGCCCATCCATAGAGGAGGACTGTCAAAAATATAT
CCTCAAACAGCAGCAGGAAGAAGCTGAAAAACCTCTGCAGGTCGCGGCTG
TCGACAGCAGCGTACCTCGAACAGCTGAGTTGGCGGGAATAACAACACTG
GATGATCCGCTTGGG.

In some embodiments, a polynucleotide provided herein comprises a nucleic acid sequence that is at least 80%, at least 85%, at least 90%, at least 91%, at least 92%, at least 93%, at least 94%, at least 95%, at least 96%, at least 97%, at least 98%, or at least 99% identical to the sequence set forth in SEQ ID NO: 5. In some embodiments, a polynucleotide provided herein comprises the nucleic acid sequence set forth in SEQ ID NO: 5. In some embodiments, a polynucleotide provided herein comprises a nucleic acid sequence that is at least 80%, at least 85%, at least 90%, at least 91%, at least 92%, at least 93%, at least 94%, at least 95%, at least 96%, at least 97%, at least 98%, or at least 99% identical to the sequence set forth in SEQ ID NO: 6. In some embodiments, a polynucleotide provided herein comprises the nucleic acid sequence set forth in SEQ ID NO: 6.

An illustrative DNA sequence encoding a CD3ζ domain fused to myD88 domain is shown in SEQ ID NO: 10, with the CD3ζ domain underlined, and the MyD88 domain shown in bold. Shown in italics is a linker sequence.

(SEQ ID NO: 10)
AGAGTGAAGTTCAGCAGGAGCGCAGACGCCCCCGCGTACCAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGCAG
AGAAGGAAGAACCCTCAGGAAGGCCTGTACAATGAACTGCAGAAAGATAA
GATGGCGGAGGCCTACAGTGAGATTGGGATGAAAGGCGAGCGCCGGAGGG
GCAAGGGGCACGATGGCCTTTACCAGGGTCTCAGTACAGCCACCAAGGAC
ACCTACGACGCCCTTCACATGCAGGCCCTGCCCCCTCGCGCGGCCGCAAT
GGCAGCAGGAGGACCAGGCGCCGGCAGCGCCGCCCCCGTGAGCTCTACCA
GCTCTCTGCCTTTAGCCGCCCTGAACATGCGCGTGAGAAGGCGCCTGAGC
CTGTTTCTGAATGTGCGGACTCAGGTCGCCGCCGACTGGACAGCCCTGGC
TGAAGAAATGGATTTTGAGTACCTGGAGATCCGCCAATTAGAGACACAGG
CCGATCCAACAGGCCGGCTGCTGGATGCCTGGCAGGGCAGACCCGGAGCC
TCCGTGGGCAGGCTGCTGGAACTGCTGACTAAGCTGGGCAGAGATGATGT
GCTGCTGGAACTGGGCCCCTCTATCGAGGAGGACTGTCAGAAATACATCC
TGAAGCAACAACAGGAAGAAGCAGAGAAGCCTCTGCAGGTGGCAGCCGTG
GATTCTAGCGTGCCAAGGACAGCCGAACTGGCCGGCATTACCACACTGGA
TGATCCTCTGGGA.

In some embodiments, a polynucleotide provided herein comprises a nucleic acid sequence that is at least 80%, at least 85%, at least 90%, at least 91%, at least 92%, at least 93%, at least 94%, at least 95%, at least 96%, at least 97%, at least 98%, or at least 99% identical to the sequence set forth in SEQ ID NO: 10. In some embodiments, a polynucleotide provided herein comprises the nucleic acid sequence set forth in SEQ ID NO: 10.

Another illustrative DNA sequence encoding a CD3ζ domain fused to myD88 domain is shown in SEQ ID NO: 15, with the CD3 domain underlined, and the MyD88 domain shown in bold.

(SEQ ID NO: 15)
ATGAAGTGGAAGGCGCTTTTCACCGCGGCCATCCTGCAGGCACAGTTGCC
GATTACAGAGGCACAGAGCTTTGGCCTGCTGGATCCCAAACTCTGCTACC
TGCTGGATGGAATCCTCTTCATCTATGGTGTCATTCTCACTGCCTTGTTC
CTGAGAGTGAAGTTCAGCAGGAGCGCAGACGCCCCCGCGTACCAGCAGGG
CCAGAACCAGCTCTATAACGAGCTCAATCTAGGACGAAGAGAGGAGTACG
ATGTTTTGGACAAGAGACGTGGCCGGGACCCTGAGATGGGGGGAAAGCCG
CAGAGAAGGAAGAACCCTCAGGAAGGCCTGTACAATGAACTGCAGAAAGA
TAAGATGGCGGAGGCCTACAGTGAGATTGGGATGAAAGGCGAGCGCCGGA
GGGGCAAGGGGCACGATGGCCTTTACCAGGGTCTCAGTACAGCCACCAAG
GACACCTACGACGCCCTTCACATGCAGGCCCTGCCCCCTCGCATGCATGT
CGACATGGCAGCAGGAGGACCAGGCGCCGGCAGCGCCGCCCCCGTGAGCT
CTACCAGCTCTCTGCCTTTAGCCGCCCTGAACATGCGCGTGAGAAGGCGC
CTGAGCCTGTTTCTGAATGTGCGGACTCAGGTCGCCGCCGACTGGACAGC
CCTGGCTGAAGAAATGGATTTTGAGTACCTGGAGATCCGCCAATTAGAGA
CACAGGCCGATCCAACAGGCCGGCTGCTGGATGCCTGGCAGGGCAGACCC
GGAGCCTCCGTGGGCAGGCTGCTGGAACTGCTGACTAAGCTGGGCAGAGA
TGATGTGCTGCTGGAACTGGGCCCCTCTATCGAGGAGGACTGTCAGAAAT
ACATCCTGAAGCAACAACAGGAAGAAGCAGAGAAGCCTCTGCAGGTGGCA
GCCGTGGATTCTAGCGTGCCAAGGACAGCCGAACTGGCCGGCATTACCAC
ACTGGATGATCCTCTGGGAGTCGACACGCGTAGAGCCAAGAGA.

In some embodiments, a polynucleotide provided herein comprises a nucleic acid sequence that is at least 80%, at least 85%, at least 90%, at least 91%, at least 92%, at least 93%, at least 94%, at least 95%, at least 96%, at least 97%, at least 98%, or at least 99% identical to the sequence set forth in SEQ ID NO: 15. In some embodiments, a polynucleotide provided herein comprises the nucleic acid sequence set forth in SEQ ID NO: 15.

As would be appreciated by a person of skill in the art, the polynucleotides described herein may comprise additional portions of a TCR or a CAR. Alternatively, a person of skill in the art would be able to obtain the sequence of a modified TCR or a CAR and further modified such a receptor to add a MyD88 domain provided herein.

In some embodiments, a polynucleotide further comprises a T2A self-cleaving fragment.

In another aspect, provided herein is a polynucleotide comprising a nucleic acid sequence encoding a CD3ζ domain fused to a MyD88 domain. An exemplary nucleic acid sequence encoding CD3ζ, a MyD88 domain and a T2A domain is set forth in SEQ D NO: 7, with the nucleic acid encoding the CD3ζ domain underlined, and the nucleic acid encoding the MyD88 domain shown in bold. Shown in italics is a nucleic acid encoding a T2A self-cleaving fragment that may be included to facilitate transfection and expression of the modified TCR

(SEQ ID NO: 7)
ATGAAGTGGAAGGCGCTTTTCACCGCGGCCATCCTGCAGGCACAGTTGCC
GATTACAGAGGCACAGAGCTTTGGCCTGCTGGATCCCAAACTCTGCTACC
TGCTGGATGGAATCCTCTTCATCTATGGTGTCATTCTCACTGCCTTGTTC
CTGAGAGTGAAGTTCAGCAGGAGCGCAGACGCCCCCGCGTACCAGCAGGG
CCAGAACCAGCTCTATAACGAGCTCAATCTAGGACGAAGAGAGGAGTACG
ATGTTTTGGACAAGAGACGTGGCCGGGACCCTGAGATGGGGGGAAAGCCG
CAGAGAAGGAAGAACCCTCAGGAAGGCCTGTACAATGAACTGCAGAAAGA
TAAGATGGCGGAGGCCTACAGTGAGATTGGGATGAAAGGCGAGCGCCGGA
GGGGCAAGGGGCACGATGGCCTTTACCAGGGTCTCAGTACAGCCACCAAG
GACACCTACGACGCCCTTCACATGCAGGCCCTGCCCCCTCGCATGCATGT
CGACATGGCAGCAGGAGGACCAGGCGCCGGCAGCGCCGCCCCCGTGAGCT
CTACCAGCTCTCTGCCTTTAGCCGCCCTGAACATGCGCGTGAGAAGGCGC
CTGAGCCTGTTTCTGAATGTGCGGACTCAGGTCGCCGCCGACTGGACAGC
CCTGGCTGAAGAAATGGATTTTGAGTACCTGGAGATCCGCCAATTAGAGA
CACAGGCCGATCCAACAGGCCGGCTGCTGGATGCCTGGCAGGGCAGACCC
GGAGCCTCCGTGGGCAGGCTGCTGGAACTGCTGACTAAGCTGGGCAGAGA
TGATGTGCTGCTGGAACTGGGCCCCTCTATCGAGGAGGACTGTCAGAAAT
ACATCCTGAAGCAACAACAGGAAGAAGCAGAGAAGCCTCTGCAGGTGGCA
GCCGTGGATTCTAGCGTGCCAAGGACAGCCGAACTGGCCGGCATTACCAC
ACTGGATGATCCTCTGGGAGTCGACACGCGTAGAGCCAAGAGAGAGGGCA
GGGGCTCTCTGCTGACATGCGGCGATGTGGAGGAGAATCCCGGCCCT

In some embodiments, a polynucleotide provided herein comprises a nucleic acid sequence that is at least 80%, at least 85%, at least 90%, at least 91%, at least 92%, at least 93%, at least 94%, at least 95%, at least 96%, at least 97%, at least 98%, or at least 99% identical to the sequence set forth in SEQ ID NO: 7. In some embodiments, a polynucleotide provided herein comprises the nucleic acid sequence set forth in SEQ ID NO: 7.

In another aspect, provided herein is a polynucleotide comprising a nucleic acid sequence encoding a CD8a domain linked to a MyD88 domain. An exemplary nucleic acid sequence encoding a CD8a, domain a MyD88 domain and linker is set forth in SEQ D NO: 8, with the nucleic acid encoding the CD8a domain underlined, the nucleic acid encoding the MyD88 domain shown in bold, and the nucleic acid encoding the G4S linker connecting the MyD88 domain and the CD8a domain shown in italics:

(SEQ ID NO: 8)
ATGGCCTTGCCGGTCACAGCCCTGCTTCTCCCACTGGCCCTTCTCCTTCA
TGCGGCGCGGCCGAGTCAGTTCAGAGTTTCACCCCTTGACAGAACCTGGA
ACCTCGGCGAGACAGTCGAGCTGAAATGCCAAGTGTTGCTTAGCAATCCA
ACTTCCGGATGCTCATGGTTGTTCCAACCACGGGGCGCCGCAGCCTCACC
AACCTTTCTGTTGTACTTGTCACAGAACAAACCTAAGGCAGCGGAAGGAC
TCGATACGCAAAGATTCAGCGGAAAGCGCTTGGGGGACACATTTGTTTTG
ACGTTGTCTGATTTCAGACGCGAAAATGAGGGGTACTATTTCTGTAGTGC
GCTTAGTAACAGTATTATGTACTTTTCTCATTTTGTGCCTGTATTCTTGC
CAGCAAAGCCCACGACCACTCCTGCCCCCCGACCTCCCACACCAGCGCCA
ACCATTGCGTCTCAACCTCTTAGCTTGCGCCCTGAAGCATGTCGGCCAGC
AGCCGGAGGTGCGGTGCATACAAGAGGTCTCGACTTCGCGTGCGATATCT
ACATCTGGGCTCCCCTGGCTGGAACCTGCGGGGTGCTCTTGTTGTCCCTT
GTCATCACATTGTATTGCAATCACAGGAATAGACGAAGAGTGTGCAAGTG
TCCGCGACCAGTGGTAAAATCAGGTGATAAACCAAGTCTTAGCGCTCGGT
ACGTGGGTGGTGGCGGCTCTGGAGGGGGTGGGAGCATGGCAGCAGGGGGC
CCTGGAGCTGGAAGTGCCGCACCAGTAAGTAGCACTTCCAGTCTTCCCCT
CGCGGCACTGAATATGAGAGTTCGCCGGCGGTTGAGCCTCTTCTTGAATG
TTAGAACGCAAGTGGCTGCTGACTGGACAGCCCTCGCGGAGGAAATGGAT
TTCGAATATTTGGAGATTAGGCAATTGGAGACTCAAGCAGATCCGACAGG
TCGCCTTCTTGACGCTTGGCAGGGTCGGCCGGGCGCTAGTGTGGGACGAC
TGCTTGAGTTGCTGACCAAATTGGGCCGGGACGATGTTCTCTTGGAGCTT
GGCCCATCCATAGAGGAGGACTGTCAAAAATATATCCTCAAACAGCAGCA
GGAAGAAGCTGAAAAACCTCTGCAGGTCGCGGCTGTCGACAGCAGCGTAC
CTCGAACAGCTGAGTTGGCGGGAATAACAACACTGGATGATCCGCTTGGG.

In some embodiments, a polynucleotide provided herein comprises a nucleic acid sequence that is at least 80%, at least 85%, at least 90%, at least 91%, at least 92%, at least 93%, at least 94%, at least 95%, at least 96%, at least 97%, at least 98%, or at least 99% identical to the sequence set forth in SEQ ID NO: 8. In some embodiments, a polynucleotide provided herein comprises the nucleic acid sequence set forth in SEQ ID NO: 8.

An illustrative nucleic acid sequence encoding a modified TCR comprising a TCR α chain, a TCR β chain, a CD3ζ intracellular domain (ICD), and a MyD88 DD is set forth in SEQ ID NO: 12, with the TCR α chain underlined, the TCR β chain shown in bold, the CD3ζ ICD shown in underlined and bold, and the MyD88 DD shown in bold italics. Shown in italics are linker sequences.

(SEQ ID NO: 12)
atgaaatccttgagagttttactagtgatcctgtggcttcagttgagctg
ggtttggagccaacagaaggaggtggagcaaAATTCtggacccctcagtg
ttccagagggagccattgcctctctcaactgcacttacagtgaccgaggt
tcccagtccttcttctggtacagacaatattctgggaaaagccctgagtt
gataatcttcatatactccaatggtgacaaagaagatggaaggtttacag
cacagctcaataaagccagccagtatgtttctctgctcatcagagactcc
cagcccagtgattcagccacctacctctgtgccgtgaacttcggaggagg
aaagcttatcttcggacagggaacggagttatctgtgaaacccaatatcc
agaaccctgaccctgccgtgtaccagctgagagactctaaatccagtgac
aagtctgtctgcctattcaccgattttgattctcaaacaaatgtgtcaca
aagtaaggattctgatgtgtatatcacagacaaaactgtgctagacatga
ggtctatggacttcaagagcaacagtgctgtggcctggagcaacaaatct
gactttgcatgtgcaaacgccttcaacaacagcattattccagaagacac
cttcttccccagcccagaaagttcctgtgatgtcaagctggtcgagaaaa
gctttgaaacagatacgaacctaaactttcaaaacctgtcagtgattggg
ttccgaatcctcctcctgaaggtggccgggtttaatctgctcatgacgct
gcggctgtggtccagcttaattaaaGTCGACGAATTCTTCGAAagagcca
aaagagagggcagaggaagtcttctaacatgcggtgacgtggaggagaat
cccggccctatgagaatcaggctcctgtgctgtgtggccttttctctcct
gtgggcaggtccagtgattgctgggatcacccaggcaccaacatctcaga
tcctggcagcaggacggcgcatgacactgagatgtacccaggatatgaga
cataatgccatgtactggtatagacaagatctaggactggggctaaggct
catccattattcaaatactgcaggtaccactggcaaaggagaagtccctg
atggttatagtgtctccagagcaaacacagatgatttccccctcacgttg
gcgtctgctgtaccctctcagacatctgtgtacttctgtgccagcagcct
aagtttcggcactgaagctttctttggacaaggcaccagactcacagttg
tagaggacctgaacaaggtgttcccacccgaggtcgctgtgtttgagcca
tcagaagcagagatctcccacacccaaaaggccacactggtgtgcctggc
cacaggcttcttccctgaccacgtggagctgagctggtgggtgaatggga
aggaggtgcacagtggggtcagcacggacccgcagcccctcaaggagcag
cccgccctcaatgactccagatactgcctgagcagccgcctgagggtctc
ggccaccttctggcagaacccccgcaaccacttccgctgtcaagtccagt
tctacgggctctcggagaatgacgagtggacccaggatagggccaaaccc
gtcacccagatcgtcagcgccgaggcctggggtagagcagactgtggctt
tacctcggtgtcctaccagcaaggggtcctgtctgccaccatcctctatg
agatcctgctagggaaggccaccctgtatgctgtgctggtcagcgccctt
gtgttgatggccatggtcaagagaaaggatTCCAGAGGCATGCATggtgg
aggtggaagtAGAGTGAAGTTCAGCAGGAGCGCAGACGCCCCCGCGTACC
AGCAGGGCCAGAACCAGCTCTATAACGAGCTCAATCTAGGACGAAGAGAG
GAGTACGATGTTTTGGACAAGAGACGTGGCCGGGACCCTGAGATGGGGGG
AAAGCCGCAGAGAAGGAAGAACCCTCAGGAAGGCCTGTACAATGAACTGC
AGAAAGATAAGATGGCGGAGGCCTACAGTGAGATTGGGATGAAAGGCGAG
CGCCGGAGGGGCAAGGGGCACGATGGCCTTTACCAGGGTCTCAGTACAGC
CACCAAGGACACCTACGACGCCCTTCACATGCAGGCCCTGCCCCCTCGCG
CGGCCGCAATGGCAGCAGGAGGACCAGGCGCCGGCAGCGCCGCCCCCGTG
AGCTCTACCAGCTCTCTGCCTTTAGCCGCCCTGAACATGCGCGTGAGAAG
GCGCCTGAGCCTGTTTCTGAATGTGCGGACTCAGGTCGCCGCCGACTGGA
CAGCCCTGGCTGAAGAAATGGATTTTGAGTACCTGGAGATCCGCCAATTA
GAGACACAGGCCGATCCAACAGGCCGGCTGCTGGATGCCTGGCAGGGCAG
ACCCGGAGCCTCCGTGGGCAGGCTGCTGGAACTGCTGACTAAGCTGGGCA
GAGATGATGTGCTGCTGGAACTGGGCCCCTCTATCGAGGAGGACTGTCAG
AAATACATCCTGAAGCAACAACAGGAAGAAGCAGAGAAGCCTCTGCAGGT
GGCAGCCGTGGATTCTAGCGTGCCAAGGACAGCCGAACTGGCCGGCATTA
CCACACTGGATGATCCTCTGGGA.

In some embodiments, a polynucleotide provided herein comprises a nucleic acid sequence that is at least 80%, at least 85%, at least 90%, at least 91%, at least 92%, at least 93%, at least 94%, at least 95%, at least 96%, at least 97%, at least 98%, or at least 99% identical to the sequence set forth in SEQ ID NO: 12. In some embodiments, a polynucleotide provided herein comprises the nucleic acid sequence set forth in SEQ ID NO: 12. An illustrative DNA sequence encoding a CD3ζ domain fused to a MyD88 domain is shown in SEQ ID NO: 14, with the CD3ζ domain underlined, and the MyD88 domain shown in bold.

(SEQ ID NO: 14)
ATGAAGTGGAAGGCGCTTTTCACCGCGGCCATCCTGCAGGCACAGTTGCC
GATTACAGAGGCACAGAGCTTTGGCCTGCTGGATCCCAAACTCTGCTACC
TGCTGGATGGAATCCTCTTCATCTATGGTGTCATTCTCACTGCCTTGTTC
CTGAGAGTGAAGTTCAGCAGGAGCGCAGACGCCCCCGCGTACCAGCAGGG
CCAGAACCAGCTCTATAACGAGCTCAATCTAGGACGAAGAGAGGAGTACG
ATGTTTTGGACAAGAGACGTGGCCGGGACCCTGAGATGGGGGGAAAGCCG
CAGAGAAGGAAGAACCCTCAGGAAGGCCTGTACAATGAACTGCAGAAAGA
TAAGATGGCGGAGGCCTACAGTGAGATTGGGATGAAAGGCGAGCGCCGGA
GGGGCAAGGGGCACGATGGCCTTTACCAGGGTCTCAGTACAGCCACCAAG
GACACCTACGACGCCCTTCACATGCAGGCCCTGCCCCCTCGCATGCATGT
CGACATGGCAGCAGGAGGACCAGGCGCCGGCAGCGCCGCCCCCGTGAGCT
CTACCAGCTCTCTGCCTTTAGCCGCCCTGAACATGCGCGTGAGAAGGCGC
CTGAGCCTGTTTCTGAATGTGCGGACTCAGGTCGCCGCCGACTGGACAGC
CCTGGCTGAAGAAATGGATTTTGAGTACCTGGAGATCCGCCAATTAGAGA
CACAGGCCGATCCAACAGGCCGGCTGCTGGATGCCTGGCAGGGCAGACCC
GGAGCCTCCGTGGGCAGGCTGCTGGAACTGCTGACTAAGCTGGGCAGAGA
TGATGTGCTGCTGGAACTGGGCCCCTCTATCGAGGAGGACTGTCAGAAAT
ACATCCTGAAGCAACAACAGGAAGAAGCAGAGAAGCCTCTGCAGGTGGCA
GCCGTGGATTCTAGCGTGCCAAGGACAGCCGAACTGGCCGGCATTACCAC
ACTGGATGATCCTCTGGGAGTCGACACGCGTAGAGCCAAGAGA.

In another aspect, provided herein are vectors comprising the polynucleotides described herein. In some embodiments, the viral vector is a non-integrating non-chromosomal vector. Exemplary non-integrating non-chromosomal vectors include, but are not limited to, adeno-associated virus (AAV), adenovirus, and herpes viruses. In some embodiments, the viral vector is an integrating chromosomal vector. Integrating chromosomal vectors include, but are not limited to, adeno-associated vectors (AAV), Lentiviruses, and gamma-retroviruses.

Lentiviruses are complex retroviruses, which, in addition to the common retroviral genes gag, pol, and env, contain other genes with regulatory or structural function. Lentiviral vectors are well known in the art (see, for example, U.S. Pat. Nos. 6,013,516 and 5,994,136).

A retroviral vector may also be, e.g., a gammaretroviral vector. A gammaretroviral vector may include, e.g., a promoter, a packaging signal (ψ), a primer binding site (PBS), one or more (e.g., two) long terminal repeats (LTR), and a transgene of interest, e.g., a gene encoding a CAR. A gammaretroviral vector may lack viral structural gens such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, e.g., in Tobias Maetzig et al., Viruses. 2011 June; 3(6): 677-713.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell—wherein a suitable host cell is transfected with two or more vectors carrying the packaging functions, namely gag, pol and env, as well as rev and tat—is described in U.S. Pat. No. 5,994,136, incorporated herein by reference.

### Cells

In another aspect, provided herein are therapeutic cells comprising the modified receptors and fusions proteins described herein. Such cells may be used in adoptive cell therapies to treat a variety of diseases and disorders, including cancer.

In some embodiments, the cell is a modified T cell comprising a modified TCR described herein. In some embodiments, the cell is a modified T cell comprising a fusion protein described herein. In some embodiments, the cell is a tumor infiltrating lymphocyte (TIL).

In one aspect, provided herein is a cell comprising (a) a modified T cell receptor (TCR) comprising an α chain, a β chain, a CD3ζ, intracellular domain and a MyD88 polypeptide comprising the death domain (DD) of MyD88 and the intermediate domain (ID) of MyD88, wherein the MyD88 domain is fused to the CD3ζ. subunit, or (b) a fusion protein comprising a CD3ζ, intracellular domain and a MyD88 DD. In some embodiments, the MyD88 polypeptide comprises an amino acid sequence that is at least 90% identical to the sequence set forth in SEQ ID NO: 1 or 2. In some embodiments, the fusion protein comprises an amino acid sequence that is at least 90% identical to the amino acid sequence set forth in SEQ ID NO: 3 or 9. In some embodiments, other T cell receptor submits are co-expressed with the fusion protein. In some embodiments, a CD3γ subunit, a CD3ζ subunit, and/or a CD3ε subunit are co-expressed with the fusion protein provided herein.

In another aspect, provided herein is a cell comprising (a) a modified T cell receptor (TCR) comprising an α chain, a β chain, a CD3ζ intracellular domain and a MyD88 polypeptide comprising an amino acid sequence that is at least 90% identical to the sequence set forth in SEQ ID NO: 1 or 2, wherein the MyD88 domain is fused to the CD3ζ subunit, or (b) a fusion protein comprising an amino acid sequence that is at least 90% identical to the amino acid sequence set forth in SEQ ID NO: 3 or 9.

In some embodiments, the cell is a modified immune cell comprising a modified TCR or a fusion protein described herein. The immune cells may be T cells (e.g., regulatory T cells, CD4⁺ T cells, CD8⁺ T cells, or gamma-delta T cells), NK cells, invariant NK cells, NKT cells, stem cells (e.g., mesenchymal stem cells (MSCs) or induced pluripotent stem (iPSC) cells) or a tumor-infiltrating lymphocyte (TIL). In some embodiments, the cells are monocytes or granulocytes, e.g., myeloid cells, macrophages, neutrophils, dendritic cells, mast cells, eosinophils, and/or basophils.

The immune cells may be isolated from subjects, particularly human subjects. The immune cells can be obtained from a subject of interest, such as a subject suspected of having a particular disease or condition, a subject suspected of having a predisposition to a particular disease or condition, or a subject who is undergoing therapy for a particular disease or condition. The immune cells may be enriched/purified from any tissue where they reside including, but not limited to, blood (including blood collected by blood banks or cord blood banks), spleen, bone marrow, tissues removed and/or exposed during surgical procedures, and tissues obtained via biopsy procedures. Tissues/organs from which the immune cells are enriched, isolated, and/or purified may be isolated from both living and non-living subjects, wherein the non-living subjects are organ donors. The isolated immune cells may be used directly, or they can be stored for a period of time, such as by freezing. In some embodiments, the immune cells are isolated from blood, such as peripheral blood or cord blood. In some embodiments, immune cells isolated from cord blood have enhanced immunomodulation capacity, such as measured by CD4-positive or CD8-positive T cell suppression. In specific aspects, the immune cells are isolated from pooled blood, particularly pooled cord blood, for enhanced immunomodulation capacity. The pooled blood may be from 2 or more sources, such as 3, 4, 5, 6, 7, 8, 9, 10 or more sources (e.g., donor subjects).

The population of immune cells can be obtained from a subject in need of therapy or suffering from a disease associated with reduced immune cell activity. Thus, the cells may be autologous to the subject in need of therapy. Alternatively, the population of immune cells can be obtained from a donor. The immune cell population can be harvested from the peripheral blood, cord blood, bone marrow, spleen, or any other organ/tissue in which immune cells reside in said subject or donor. The immune cells can be isolated from a pool of subjects and/or donors, such as from pooled cord blood. The population of immune cells can be derived from induced pluripotent stem cells (iPSCs) and/or any other stem cell known in the art. In some aspects, the iPSCs and/or stem cells used to derive the population of immune cells can be obtained from a subject in need of therapy or suffering from a disease associate with reduced immune cell activity, thus these IPSCs and/or stem cells will be autologous to the subject in need of therapy. Alternatively, the iPSCs and/or stem cells can be obtained from a donor and therefore be allogeneic to the subject in need of therapy.

When the population of immune cells is obtained from a donor distinct from the subject, the donor is preferably allogeneic, provided the cells obtained are subject-compatible in that they can be introduced into the subject. Allogeneic donor cells are may or may not be human leukocyte antigen (HLA)-compatible. To be rendered subject-compatible, allogeneic cells can be treated to reduce immunogenicity.

Cells may be modified to express the modified receptors or fusion proteins described herein by any suitable method known in the art or described herein, for example, electroporation or lipofection. In some embodiments of the methods of the disclosure, introducing a nucleic acid sequence and/or a genomic editing construct into an immune cell ex vivo, in vivo, in vitro or in situ comprises a viral vector. In some embodiments of the methods of the disclosure, introducing a nucleic acid sequence and/or a genomic editing construct into an immune cell ex vivo, in vivo, in vitro or in situ comprises a combination of vectors. Exemplary, non-limiting vector combinations include: viral and non-viral vectors, a plurality of non-viral vectors, or a plurality of viral vectors. Exemplary but non-limiting vectors combinations include: a combination of a DNA-derived and an RNA-derived vector, a combination of an RNA and a reverse transcriptase, a combination of a transposon and a transposase, a combination of a non-viral vector and an endonuclease, and a combination of a viral vector and an endonuclease.

In some embodiments of the methods of the disclosure, genome modification comprising introducing a nucleic acid sequence and/or a genomic editing construct into an immune cell ex vivo, in vivo, in vitro or in situ stably integrates a nucleic acid sequence, transiently integrates a nucleic acid sequence, produces site-specific integration a nucleic acid sequence, or produces a biased integration of a nucleic acid sequence. In some embodiments, the nucleic acid sequence is a transgene.

In some embodiments of the methods of the disclosure, genome modification comprising introducing a nucleic acid sequence and/or a genomic editing construct into an immune cell ex vivo, in vivo, in vitro or in situ stably integrates a nucleic acid sequence. In some embodiments, the stable chromosomal integration can be a random integration, a site-specific integration, or a biased integration. In some embodiments, the site-specific integration can be non-assisted or assisted. In some embodiments, the assisted site-specific integration is co-delivered with a site-directed nuclease. In some embodiments, the site-directed nuclease comprises a transgene with 5′ and 3′ nucleotide sequence extensions that contain a percentage homology to upstream and downstream regions of the site of genomic integration. In some embodiments, the transgene with homologous nucleotide extensions enable genomic integration by homologous recombination, microhomology-mediated end joining, or nonhomologous end-joining. In some embodiments the site-specific integration occurs at a safe harbor site. Genomic safe harbor sites are able to accommodate the integration of new genetic material in a manner that ensures that the newly inserted genetic elements function reliably (for example, are expressed at a therapeutically effective level of expression) and do not cause deleterious alterations to the host genome that cause a risk to the host organism. Potential genomic safe harbors include, but are not limited to, intronic sequences of the human albumin gene, the adeno-associated virus site 1 (AAVS1), a naturally occurring site of integration of AAV virus on chromosome 19, the site of the chemokine (C-C motif) receptor 5 (CCR5) gene and the site of the human ortholog of the mouse Rosa26 locus.

In some embodiments, the site-specific transgene integration occurs at a site that disrupts expression of a target gene. In some embodiments, disruption of target gene expression occurs by site-specific integration at introns, exons, promoters, genetic elements, enhancers, suppressors, start codons, stop codons, and response elements. In some embodiments, exemplary target genes targeted by site-specific integration include but are not limited to any immunosuppressive gene, and genes involved in allo-rejection.

In some embodiments, the site-specific transgene integration occurs at a site that results in enhanced expression of a target gene. In some embodiments, enhancement of target gene expression occurs by site-specific integration at introns, exons, promoters, genetic elements, enhancers, suppressors, start codons, stop codons, and response elements.

In addition to viral delivery of the nucleic acids encoding the antigen receptor, the following are additional methods of recombinant gene delivery to a given cell, (e.g. an NK cell) and are thus considered in the present disclosure.

Introduction of a nucleic acid, such as DNA or RNA, into the immune cells of the current disclosure may use any suitable methods for nucleic acid delivery for transformation of a cell, as described herein or as would be known to one of ordinary skill in the art. Such methods include, but are not limited to, direct delivery of DNA such as by ex vivo transfection, by injection, including microinjection); by electroporation; by calcium phosphate precipitation; by using DEAE-dextran followed by polyethylene glycol; by direct sonic loading; by liposome mediated transfection and receptor-mediated transfection; by microprojectile bombardment; by agitation with silicon carbide fibers; by Agrobacterium-mediated transformation; by desiccation/inhibition-mediated DNA uptake, and any combination of such methods. Through the application of techniques such as these, organelle(s), cell(s), tissue(s) or organism(s) may be stably or transiently transformed.

In some embodiments of the methods of the disclosure, introducing a nucleic acid sequence and/or a genomic editing construct into an immune cell ex vivo, in vivo, in vitro or in situ comprises a non-viral vector. In some embodiments, the non-viral vector comprises a nucleic acid. In some embodiments, the non-viral vector comprises plasmid DNA, linear double-stranded DNA (dsDNA), linear single-stranded DNA (ssDNA), DoggyBone™ DNA, nanoplasmids, minicircle DNA, single-stranded oligodeoxynucleotides (ssODN), DDNA oligonucleotides, single-stranded mRNA (ssRNA), and double-stranded mRNA (dsRNA). In some embodiments, the non-viral vector comprises a transposon of the disclosure.

In some embodiments of the methods of the disclosure, enzymes may be used to create strand breaks in the host genome to facilitate delivery or integration of the transgene. In some embodiments, enzymes create single-strand breaks. In some embodiments, enzymes create double-strand breaks. In some embodiments, examples of break-inducing enzymes include but are not limited to: transposases, integrases, endonucleases, meganucleases, megaTALs, CRISPR-Cas9, CRISPR-CasX, transcription activator-like effector nucleases (TALEN) or zinc finger nucleases (ZFN). In some embodiments, break-inducing enzymes can be delivered to the cell encoded in DNA, encoded in mRNA, as a protein, as a nucleoprotein complex with a guide RNA (gRNA).

### Pharmaceutical Compositions

Also provided herein are pharmaceutical compositions comprising the cell described herein. A pharmaceutical composition may further comprise a pharmaceutically acceptable carrier. The phrases “pharmaceutical or pharmacologically acceptable” refers to molecular entities and compositions that do not produce an adverse, allergic, or other untoward reaction when administered to an animal, such as a human, as appropriate. For animal (e.g., human) administration, it will be understood that preparations should meet sterility, pyrogenicity, general safety, and purity standards as required, e.g., by the FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art (Remington's Pharmaceutical Sciences 22^{nd} edition. 2012), in the form of lyophilized formulations or aqueous solutions. The pH and exact concentration of the various components in a pharmaceutical composition are adjusted according to well-known parameters.

Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride: benzalkonium chloride; benzethonium chloride; phenol, butyl or benzyl alcohol: alkyl parabens such as methyl or propyl paraben: catechol: resorcinol; cyclohexanol; 3-pentanol; and m-cresol): low molecular weight (less than about 10 residues) polypeptides: proteins, such as serum albumin, gelatin, or immunoglobulins; hydrophilic polymers such as polyvinylpyrrolidone: amino acids such as glycine, glutamine, asparagine, histidine, arginine, or lysine; monosaccharides, disaccharides, and other carbohydrates including glucose, mannose, or dextrins; chelating agents such as EDTA: sugars such as sucrose, mannitol, trehalose or sorbitol: salt-forming counter-ions such as sodium; metal complexes (e.g. Zn— protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter Intemational, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

### Methods of Use

The MyD88 domains (e.g., the MyD88 domains of SEQ ID NOs: 1 or 2) and the fusion proteins (e.g., the fusion proteins of SEQ ID NO: 3 or 9) provided herein may be used to improve existing cellular therapies, as would be appreciated by a person of skill in the art. For example, the MyD88 domain may be fused or linked to a CD3ζ domain present in a CAR or TCR, and the fusion protein may be introduced into to an immune cell.

Exemplary engineered antigen receptors, including CARs and recombinant TCRs, as well as methods for engineering and introducing the receptors into cells, include those described, for example, in PCT Publication Nos. WO 2000/14257, WO 2013126726, WO 2012/129514, WO 2014/031687, WO 2013/166321, WO 2013/071154, and WO 2013/123061, U.S. Patent Application Publication Nos. US 2002/131960, US 2013/287748, and US 2013/0149337; and U.S. Pat. Nos. 6,451,995, 7,446,190, 8,252,592, 8,339,645, 8,398,282, 7,446,179, 6,410,319, 7,070,995, 7,265,209, 7,354,762, 7,446,190, 7,446,191, 8,324,353, and 8,479, 118; International Patent Application Publication No.: WO 2014/055668 A1, and European Patent Application Publication No. EP2537416; and/or those described by Sadelain et al., 2013; Davila et al., 2013; Turtle et al., 2012; Wu et al., 2012.

Thus, in one aspect, provided herein is a method of improving a therapeutic cell or a cell therapy, the method comprising transfecting the therapeutic cell with a modified receptor described herein. In another aspect, provided herein is a method of improving a therapeutic cell or a cell therapy, the method comprising transfecting the therapeutic cell with a fusion protein described herein.

In some embodiments, a method of improving a therapeutic cell results in an increase in expansion of the therapeutic cell in vivo, as determined by the increase in cell number over time. In some embodiments, the method of improving a therapeutic cell results in an increase in expansion of the therapeutic cell in vivo of at least 10%, at least 20%, at least 30%, at least 40%, 50%, at least 60%, at least 70%, at least 80%, at least 90%, at least 100%, at least 200%, at least 300%, at least 400%, or at least 500% compared to the expansion of therapeutic cell prior to improvement or compared to a therapeutic cell not comprising a modified TCR or a fusion protein described herein. The increase in expansion may be determined, for example, by measuring the amount of cells per volume blood at a certain time point after administration of the cell.

In some embodiments, a method of improving a therapeutic cell results in an increase in cytokine production of the therapeutic cell. Immune cells produce a variety of cytokines, including interferon gamma (INFγ), C—C Motif Chemokine Ligand 11 (CCL-11), Granulocyte-Macrophage Colony-Stimulating Factor (GM-CSF), Granzyme 9, interleukin (IL)-10, IL-12, IL-13, IL-15, IL-17A, IL-17F, IL-1b, IL-2, IL-21, IL-22, IL-4, IL-5, IL-6, IL-7, IL-8, IL-9, IP-10, Monocyte Chemoattractant Protein (MCP)-1, MCP-4, Macrophage Inflammatory Protein (MIP)-1α, MIP-1β, Perforin, Regulated Upon Activation, Normal T Cell Expressed And Presumably Secreted (RANTES), Transforming Growth Factor (TGF)-β1, Tumor Necrosis Factor (TNF)-α, TNF-β, soluble Cluster of Differentiation (sCD)137, and sCD40L. In some embodiments, the method results in an increase in the production of a cytokine in a therapeutic population of cells of at least 10%, at least 20%, at least 30%, at least 40%, 50%, at least 60%, at least 70%, at least 80%, at least 90%, at least 100%, at least 200%, at least 300%, at least 400%, or at least 500% compared to therapeutic population of cells prior to improvement or compared to a therapeutic cell not comprising a modified TCR or a fusion protein described herein. Cytokine production may be determined by measuring cytokine levels in the cell culture medium, for example, by using ELISA or Western Blotting.

In some embodiments, a method of improving a therapeutic cell described herein results in an immune cell becoming polyfunctional and expressing two or more cytokines. In some embodiments, the method of improving a therapeutic cell results in an increase in the number of cytokines product by the cell. In some embodiments, the method results in the production of at least 1, at least 2, at least 3, at 4, or at least 5 additional cytokines produced by the cell compared to a cytokine production by the therapeutic cell prior to improvement or compared to the cytokine production by a therapeutic cell not comprising a modified TCR or a fusion protein described herein.

In some embodiments, the methods produced herein may be used to improve a therapeutic population of cells, wherein the method results in a higher proportion of polyfunctional cells in the therapeutic population of cells compared to the population of cells prior to improvement or compared to a population of therapeutic cells not comprising a modified TCR or a fusion protein described herein. In some embodiments, the method results in an increase in the proportion of polyfunctional cells in a therapeutic population of cells of at least 10%, at least 20%, at least 30%, at least 40%, 50%, at least 60%, at least 70%, at least 80%, at least 90%, at least 100%, at least 200%, at least 300%, at least 400%, or at least 500% compared to therapeutic population of cells prior to improvement or compared to a therapeutic cell not comprising a modified TCR or a fusion protein described herein.

In some embodiments, the method of improving a therapeutic cell results in a decrease in exhaustion of the therapeutic cell in vivo. Immune cell exhaustion is the progressive loss of cell functionality in vivo. Immune cell exhaustion is not immediately accompanied by a decrease in cell number, but is rather measured by a decrease in the expression of certain cytokines, and the decrease in the ability of a cell effector functions. Molecular markers of cell exhaustion are known in the art and include, for example, elevated expression of inhibitory receptors (IRs), little to no proliferation, high expression of PD-1, TIM3, and LAG3 inhibitory receptors, loss of IL-2 (in early stages) and TNF (in later stages) production. Exhausted T cells may retain the ability to produce chemokines including Mip1α,Mip1β, Rantes, and IL-100. Altered epigenetic, metabolic, and transcriptional profiles, have also been described. See. e.g., McLane et al., Annu. Rev. Immunol. 2019. 37:457-95.

In some embodiments, the method results in a decrease in the proportion of exhausted cells in a therapeutic population of cells of at least 10%, at least 20%, at least 30%, at least 40%, 50%, at least 60%, at least 70%, at least 80%, at least 90%, at least 100%, at least 200%, at least 300%, at least 400%, or at least 500% compared to therapeutic population of cells prior to improvement or compared to a population of therapeutic cell not comprising a modified TCR or a fusion protein described herein.

In some embodiments, a method of improving a therapeutic cell results increases the proliferative capacity of the therapeutic cell. An increased proliferative capacity may mean, for example, that the cell continues to proliferate even after multiple rounds of simulation. In some embodiments, the method results in an increase in the proliferative capacity of a therapeutic cell of at least 10%, at least 20%, at least 30%, at least 40%, 50%, at least 60%, at least 70%, at least 80%, at least 90%, at least 100%, at least 200%, at least 300%, at least 400%, or at least 500% compared to the therapeutic cell prior to improvement or compared to a therapeutic cell not comprising a modified TCR or a fusion protein described herein.

In some embodiments, a method of improving a therapeutic cell results in an improvement in the persistence of the therapeutic cell, e.g., persistence in the blood of a subject to whom the cell has been administered. Cell persistence may be measured by monitoring the number of cells in the subject's blood, using, e.g., hemocytometer counts of flow cytometry. In some embodiments, the method results in an increase in the persistence of a therapeutic cell in the blood of a subject to whom the cells have been administered of at least 10%, at least 20%, at least 30%, at least 40%, 50%, at least 60%, at least 70%, at least 80%, at least 90%, at least 100%, at least 200%, at least 300%, at least 400%, or at least 500% compared to the therapeutic cell prior to improvement or compared to a therapeutic cell not comprising a modified TCR or a fusion protein described herein.

In some embodiments, the method of improving a therapeutic cell results in a beneficial change in the tumor microenvironment. Examples of beneficial changes in the tumor environment are known in the art and include, for example, decreases in hypoxia and decreases in vascularization. See. e.g., Benavente et al., Front. Oncol., 23 Oct. 2020, which is incorporated herein by reference in its entirety.

Preferably, the methods of improving therapeutic cells and therapeutic populations of cells do not substantially affect the antigen specificity of the receptor comprised by the cells. Thus, in preferred embodiments, a method of improving a therapeutic cell described herein results in an improved cell with substantially the same antigen specificity. “Substantially the same antigen specificity” means that the type of antigen recognized by the receptor is unchanged and that the affinity of a receptor for the antigen is decreased by at most 10% compared to the receptor of the cell prior to improvement.

The cells and pharmaceutical compositions described herein may be used to treat a disorder or disease in a subject in need thereof. Thus, is another aspect, provided herein is a method of treating cancer in a subject, comprising administering to the subject a cell comprising a modified receptor or a fusion protein provided herein. In some embodiments, the disease or disorder is cancer.

Tumors for which the present treatment methods are useful include any malignant cell type, such as those found in a solid tumor or a hematological tumor. In some embodiments, the cancer is a CD22-positive cancer. In some embodiments, the cancer has a low expression of CD22 (e.g. a CD22 low cancer). In some embodiments, the cancer is a CD19-positive cancer. In some embodiments, the cancer has a low expression of CD19 (e.g. a CD19 low cancer).

Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include but are not limited to tumors of the bone marrow, T or B cell malignancies, myeloid malignancies, leukemias, lymphomas, blastomas, myelomas. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp: adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma: infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma: teratoma, malignant; struma ovarii, malignant; choriocarcinoma; mesonephroma, malignant; hemangiosarcoma; hemangioendothelioma, malignant; kaposi's sarcoma: hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; T lymphoblastic leukemia; T lymphoblastic lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); chronic myeloid leukemia, acute lymphoblastic leukemia (ALL); acute lymphoblastic lymphoma; acute myeloid leukemia (AML); myelodysplastic syndrome (MDS); myeloproliferative neoplasms; chronic myeloblasts leukemia; diffuse large B-cell lymphoma (DLBCL); peripheral T-cell lymphoma (PTCL); or anaplastic large cell lymphoma (ALCL).

Particular embodiments concern methods of treatment of leukemia. Leukemia is a cancer of the blood or bone marrow and is characterized by an abnormal proliferation (production by multiplication) of blood cells, usually immature white blood cells (leukocytes). It is part of the broad group of diseases called hematological neoplasms. Leukemia is a broad term covering a spectrum of diseases. Leukemia is clinically and pathologically split into its acute and chronic forms and/or by and the cell type of origin (myeloid or lymphoid). In some embodiments, the leukemia is an antigen-low leukemia. In some embodiments, the leukemia is a CD22-low leukemia.

In some embodiments, the cancer is breast cancer, sarcoma, melanoma, or lung cancer. The terms “subject” and “patient” are used interchangeably herein. In some embodiments, the subject treated in accordance with the methods described herein is a human patient, e.g., a human adult.

In some embodiments, the cells are obtained from a patient, modified ex vivo, expanded, and reinfused to the patient. The cells may be allogeneic or autologous to the patient.

A person of skill in the art will be able to determine the appropriate duration and dose of treatment for a cell comprising a modified receptor or a fusion protein provided herein and/or for a pharmaceutical composition provided herein.

The cell comprising a modified receptor or a fusion protein provided herein or the pharmaceutical composition provided herein may be administered by any suitable route of administration, including, for example, intravenous, intrathecal, intraocular, subcutaneous, intraperitoneal, intramuscular, intracerebral, intraventricular, or intratracheal administration. In preferred embodiments, the cell comprising a modified receptor or a fusion protein provided herein or the pharmaceutical composition provided herein is administered intravenously.

In certain embodiments of the present disclosure, immune cells are delivered to an individual in need thereof, such as an individual that has cancer or an infection. The cells then enhance the individual's immune system to attack or directly attack the respective cancer or pathogenic cells. In some cases, the individual is provided with one or more doses of the immune cells. In cases where the individual is provided with two or more doses of the immune cells, the duration between the administrations should be sufficient to allow time for propagation in the individual, and in specific embodiments the duration between doses is 1, 2, 3, 4, 5, 6, 7, 8, 9, 10, 11, or 12 or more weeks.

In some embodiments, a subject treated in accordance with the methods described herein does not undergo lymphodepletion prior to administration of the T cell. In some embodiments, a subject treated in accordance with the methods described herein does not receive cytokines while being administered the cell.

The immune cells may be administered in combination with one or more other therapeutic agents for the treatment of the immune-mediated disorder. The immune cells may be administered before, during, after, or in various combinations relative to an additional cancer therapy, such as radiation therapy, chemotherapy, or immune therapy (e.g., immune checkpoint therapy). The administrations may be in intervals ranging from concurrently to minutes to days to weeks. In embodiments where the immune cell therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below an immune cell therapy is “A” and an anti-cancer therapy is “B”: A/B/A B/A/B B/B/A A/A/B A/B/B B/A/A A/B/B/B B/A/B/B B/B/B/A B/B/A/B A/A/B/B A/B/A/B A/B/B/A B/B/A/A B/A/B/A B/A/A/B A/A/A/B B/A/A/A A/B/A/A A/A/B/A

Administration of any compound or therapy of the present embodiments to a patient ill follow general protocols for the administration of such compounds, taking into account the toxicity, if any, of the agents. Therefore, in some embodiments there is a step of monitoring toxicity that is attributable to combination therapy.

The cell comprising a modified receptor or a fusion protein provided herein or the pharmaceutical composition provided herein may be administered for any suitable duration, for example, until symptoms improve, or for a predetermined duration such as 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, or 15 weeks.

The efficacy of a treatment for cancer may be assessed by any suitable method known in the art or described herein, including, for example, by monitoring tumor growth or measuring the time to tumor recurrence. In some embodiments, a method of treatment described herein induces tumor regression. In some embodiments, the tumor regresses by at least 10%, by at least 20%, by at least 30% within about one week, about 2 weeks, about one month, about 2 months, about 3 months, about 6 months, about 9 months, or about 12 months after the first administration of the bispecific binding agent or functional fragment thereof or of the pharmaceutical composition.

In some embodiments, a method of treatment described herein results in regression of the tumor to undetectability and delays tumor recurrence. In some embodiments, the tumor recurrence s delayed by at least about 3 months, at least about 6 months, at least about 9 months, at least about 12 months, at least 18 months, at least 24 months, at least 3 years, at least 4 years or at least 5 years after the tumor becomes undetectable.

The efficacy of a method of treatment described herein may be assessed in comparison to an untreated subject having a comparable diagnosis or to a subject having a comparable diagnosis who is receiving standard of care therapy. In some embodiments, the efficacy of a method described herein is compared to the subjected treated in accordance with a method described herein prior to the first administration.

### Kits

An article of manufacture or a kit is provided comprising the modified receptors, fusion proteins or immune cells is also provided herein. The article of manufacture or kit can further comprise a package insert comprising instructions for using the immune cells to treat or delay progression of cancer in an individual or to enhance immune function of an individual having cancer. Any of the antigen-specific immune cells described herein may be included in the article of manufacture or kits. Suitable containers include, for example, bottles, vials, bags and syringes. The container may be formed from a variety of materials such as glass, plastic (such as polyvinyl chloride or poly olefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

### EXAMPLES

The examples in this section are provided for illustration only and are not intended to limit the invention.

### Example 1: Expression of TCR:CD3z:MyD88 Constructs in Human T Cells

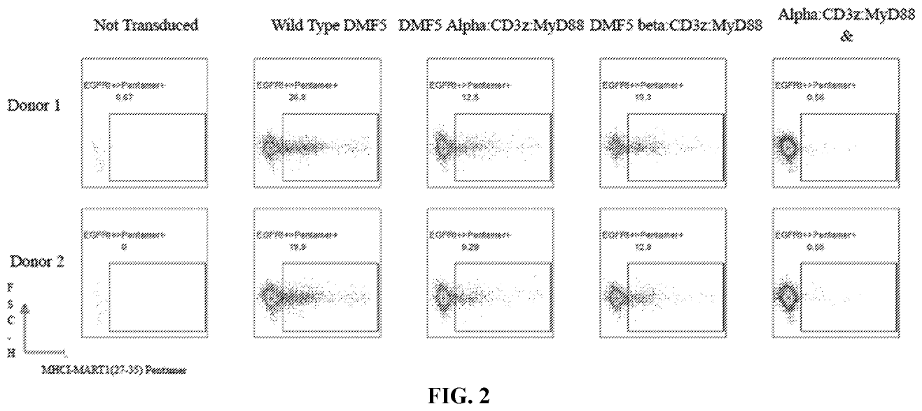

Human T cells collected from peripheral blood mononuclear cells (PBMC) were activated using soluble anti-CD3 (OKT3 clone 50 ng/ml/5E6 cells) and IL-2 (100 U/ml) for 48-72 hrs. Cells were genetically engineered using gammaretroviral particles expressing each of the indicated DMF5 TCR variants. MHC I-MART1(27-35) pentamer was used to detect the expression of the DMF5 TCR ˜72 hours after transduction using flow cytometry. Results are shown in FIG. 2. Wildtype, DMF5alpha:MyD88 and DMF5beta:MyD88 were expressed on the T cell's surface. DMF5 TCR containing the MyD88 on both the alpha and beta TCR chains was weakly detected on the T cell's surface. Rhe MHC I-MART1(27-35) pentamer bound the TCR, indicating that the TCR retains specificity to the peptide antigen and MHC I molecule.

### Example 2: T Cells Engineered with the DMF5 CD3z:MyD88 Appended to the TCRbeta or TCRalpha Chain Increases Effector Cytokine Expression by Intracellular Staining

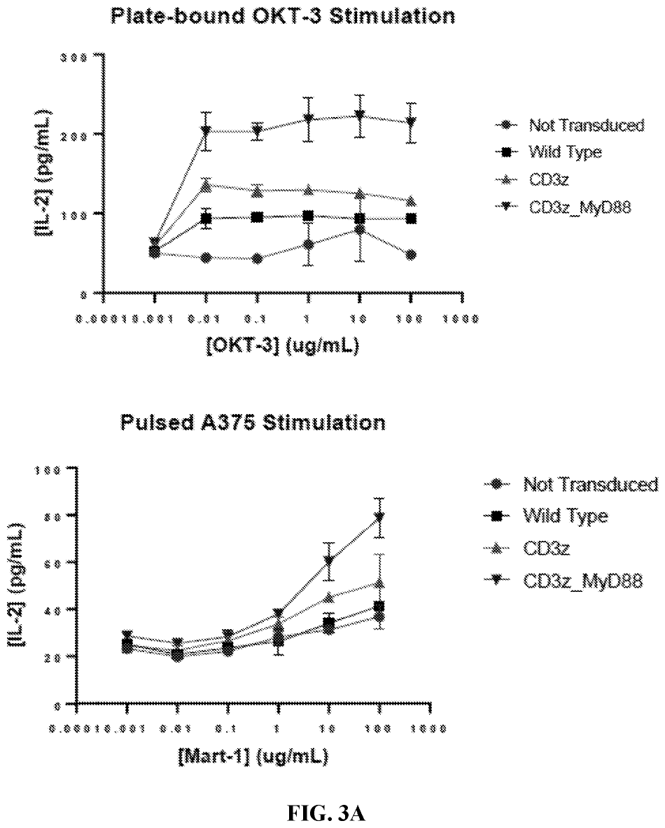

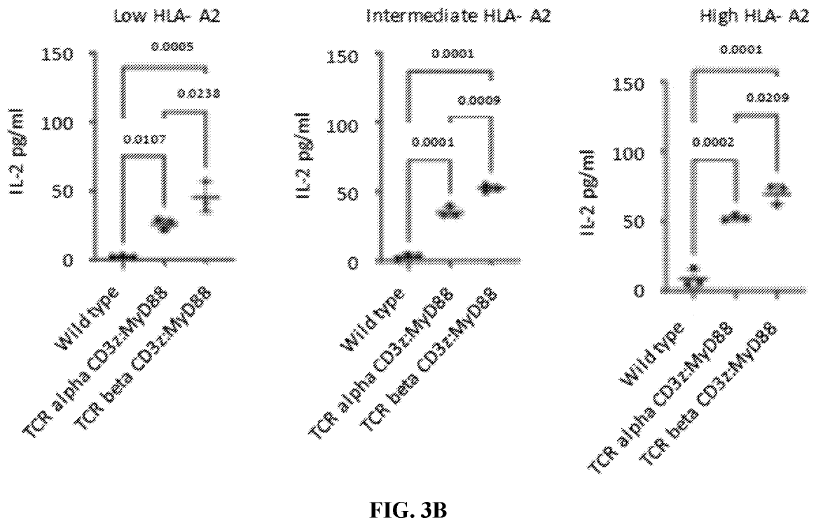

In this experiment, it was determined if appending the intracellular domain of CD3z with or without MyD88 to the TCR was functional and improved IL-2 secretion Jurkat T cells. Jurkat cells were engineered to express the DMF5 TCR CD3z:MyD88 constructs using gamma-retroviral vectors. Transduced Jurkat cells were stimulated with increasing concentrations of plate-bound anti-CD3 antibody or decreasing numbers of human melanoma HLA-A2+ A375 cells expressing MART-1(27-35) for 20 hours at which point supernatant was collected and tested for the presence of IL-2 by ELISA. A375 cells were incubated with the indicated concentrations of peptide. Results are shown in FIG. 3A, where each point represents the mean of three technical replicates, error bars=1 standard deviation. *indicates significant difference (p<0.05) between DMF5 α:CD3z:MyD88 and DMF5 β:CD3z:MyD88 groups with Wild Type DMF5 but not with each other and **indicates significant difference (p<0.05) between α:CD3z:MyD88, β:CD3z:MyD88 groups, Wild Type each with each other by one-way Anova.

624 melanoma cells (naturally expressing HLA-A2+MART1+) were sorted on varying levels of HLA-A2 expression then used to stimulate engineered Jurkat cells as described above. Results are shown in FIG. 3B, where each point represents one technical replicate, each bar represents mean of replicates, error bars=1 standard deviation. p-values are as indicated in the graphs and were determined by one-way ANOVA.

Jurkat T cells engineered with the DMF5 TCR containing CD3z:MyD88 constructs performed better than the wild type TCR. (see FIG. 3A). Jurkat T cells engineered with the all DMF5 TCR variants recognized antigen in a concentration-dependent manner when stimulated with A375 melanoma cells. (FIG. 3A right panel). Jurkat cells expressing TCR:CD3z:MyD88 variants outperformed WT TCR when stimulated with A375 cells. (FIG. 3A right panel). Jurkat T cells engineered with DMF5 TCR containing the alpha or beta CD3z:MyD88 outperformed WT TCR. However, the DMF5 beta:CD3z:MyD88 appeared to outperform the DMF5 alpha:CD3z:MyD88 when stimulated with tumor cells expressing low, medium or high MHC I levels.

### Example 3: T Cells Engineered with the TCRB:CD3z:MyD88 Demonstrate Enhanced Responses and are More Polyfunctional than Cells Engineered with Wild Type TCR or Control TCR:CD3z

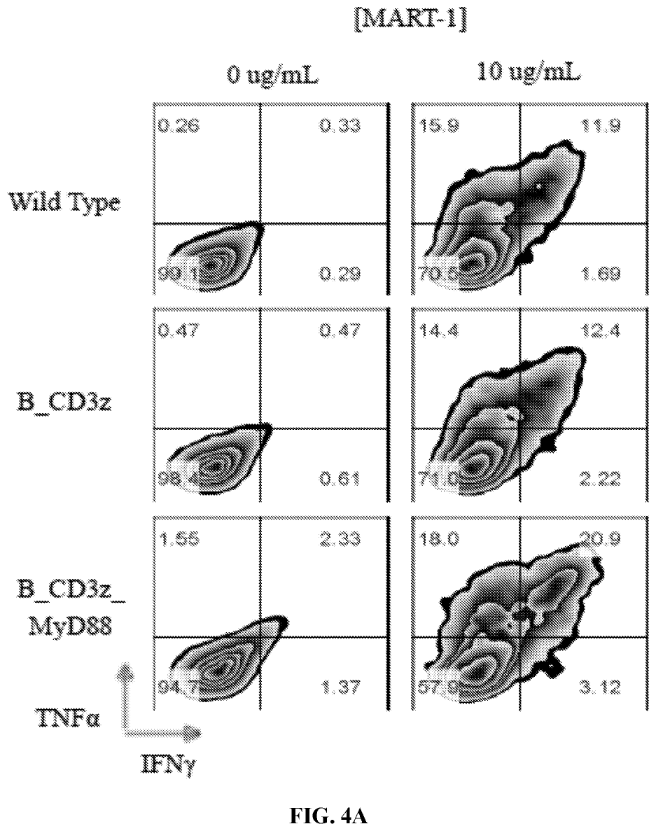

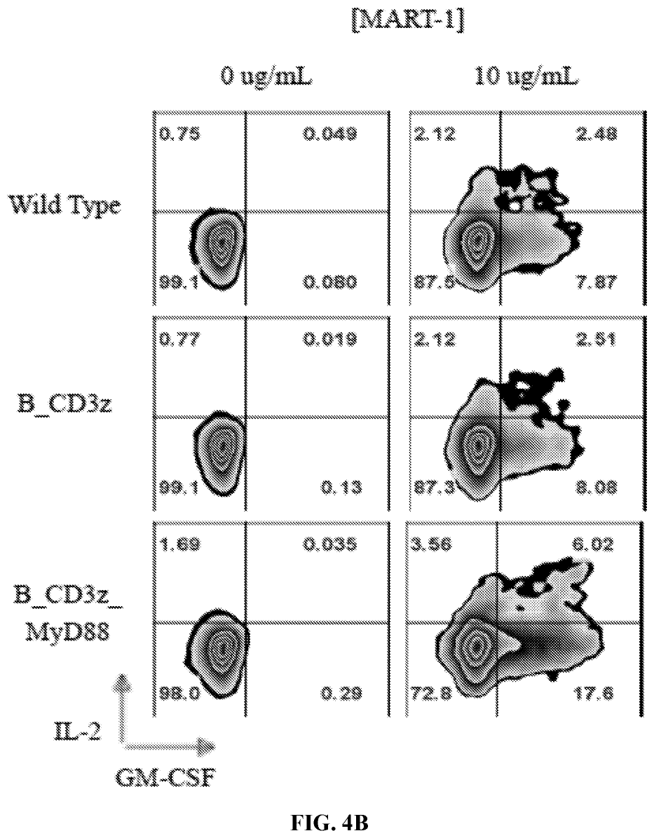

The aim of this experiments was to determine if appending the intracellular domain of CD3z with or without MyD88 to the TCR altered polyfunctionality based on cytokine production. T cells engineered with the indicated TCR constructs were pre-incubated with golgi-stop for one hour and stimulated with A375 melanoma cells pulsed with either no peptide antigen or with 10 ug/mL MART-1(27-35). After 2 hours of coculture, the stimulated T cells were collected and stained for the intracellular cytokines TNFα, IFNγ, IL-2, and GM-CSF. Results are shown in FIGS. 4A and 4B. In the absence of stimulation none of the engineered T cells produced effector molecules. This indicates that the MHC-antigen is necessary to initiate TCR responses and therefore the TCR is not overtly activated (left columns on each of FIGS. 4A and 4B). CD3z and MyD88 improved cytokine production in T cells when appended to the B chain of the TCR but not CD3z alone. A higher percentage of T cells engineered with the TCRB:CD3z:MyD88 demonstrated an ability to produce at least 2 cytokines than control groups in response to stimulation with melanoma cells. More TCRB:CD3z:MyD88 T cells produced a GM-CSF than control T cells (FIG. 4B).

### Example 4: T Cells Engineered with the TCRB:CD3z:MyD88 Demonstrate Enhanced Cytokine Production than Cells Engineered with Wild Type TCR, TCRA:CD3z:MyD88, or TCR:CD3z

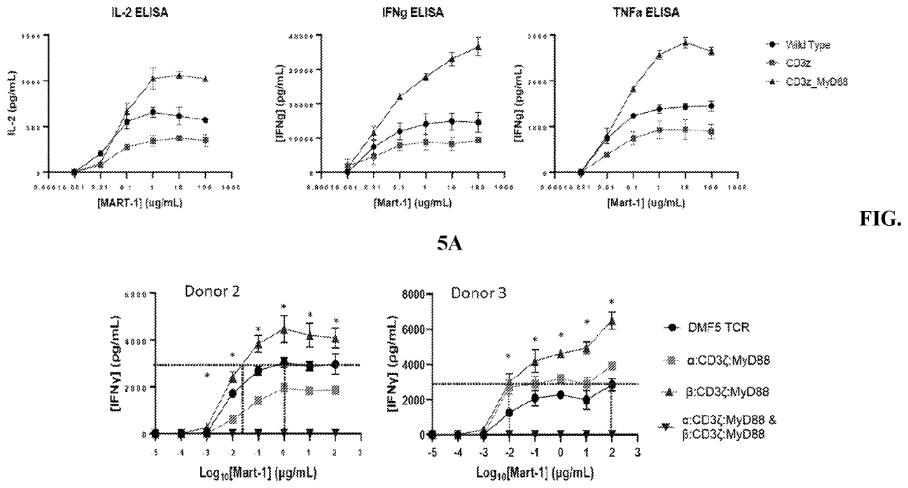

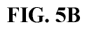

T cells extracted from human PBMCs and engineered with the various DMF5 TCR constructs were stimulated with A375 melanoma cells pulsed with increasing concentrations of MART-1(27-35) peptide. After 6 hours, the supernatant was collected and the levels of TNFa, IFNg, or IL-2 was determined by ELISA. FIG. 5A shows comparisons between DMF5 WT, TCRB:CD3z, and TCRB:CD3z:MyD88 and FIG. 5B shows comparisons between DMF5 WT, TCRB:CD3z, TCRA:CD3z:MyD88, and TCRB:CD3z:MyD88. Each dot represents the mean of three technical replicates, error bars=1 Standard Deviation.

TCRB:CD3z:MyD88-engineered T cells demonstrated an enhanced ability to produce cytokines than control T cells. Cytokine secretion occurred in an antigen concentration-dependent fashion. In the absence of antigen, T cells did not produce cytokines, highlighting that the engineered TCR retained its need to recognize MHC-antigen. TCRB:CD3z:MyD88 was more responsive than control WT or TCR:CD3z based on their ability to become stimulated by ˜10× less antigen. FIG. 5A shows that the levels of cytokines produced by TCRB:CD3z:MyD88 T cells continued to rise in response to elevated levels of antigenic stimulation. In sharp contrast, the control T cells ability to produce cytokines leveled off an ˜0.1 ug/ml of antigen. This suggests that TCRB:CD3z:MyD88 is over-riding the T cell's natural ability to suppress cytokine responses.

Collectively, the ability of a T cell to respond to suboptimal antigen levels is a beneficial feature as cancer cells can evade T cell detection by lowering the expression levels of antigen or MHC I or by expressing weakly immunogenic antigens. The ability of T cells to produce higher levels of effector molecules is a beneficial feature as T cells will eventually undergo exhaustion and reduce or shutdown cytokine production.

### Example 5: TCRB:CD3z:MyD88 Imparts T Cells Improved and Sustained Expansion Over Repeated Stimulations

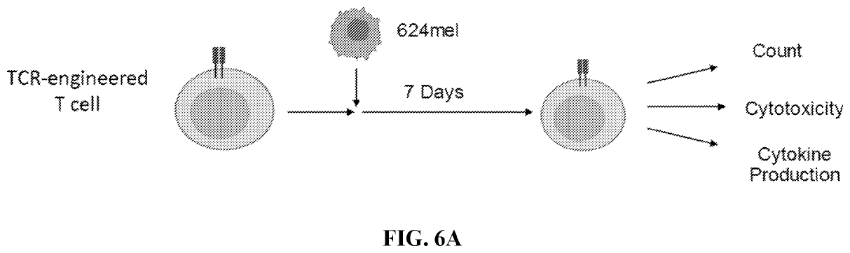

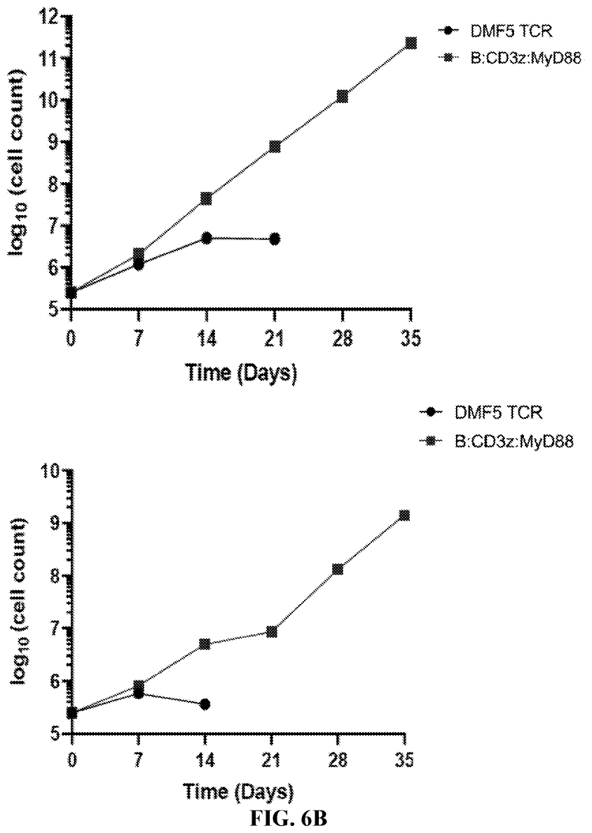

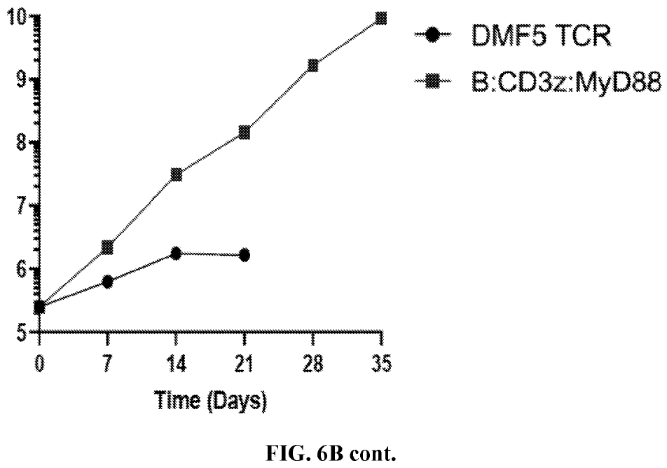

T cells were engineered with the indicated DMF5 TCRs and stimulated with HLA-A2+MART-1+624mel cells (2 tumor: 1 T cell) for 7 days at which point they were collected, counted, and restimulated. Results are shown in FIGS. 6A-6C. TCRB:CD3z:MyD88 T cells expanded at a faster rate than control T cells. The expansion of control T cells leveled off after one stimulation and reach terminal exhaustion after two stimulations (loss of ability to persist). TCRB:CD3z:MyD88 confered T cells an ability to continue to expand over repeated stimulations.

### Example 6: TCRB:CD3z:MyD88 Imparts T Cells Sustained Anti-Tumor Activity and Effector Molecule Production

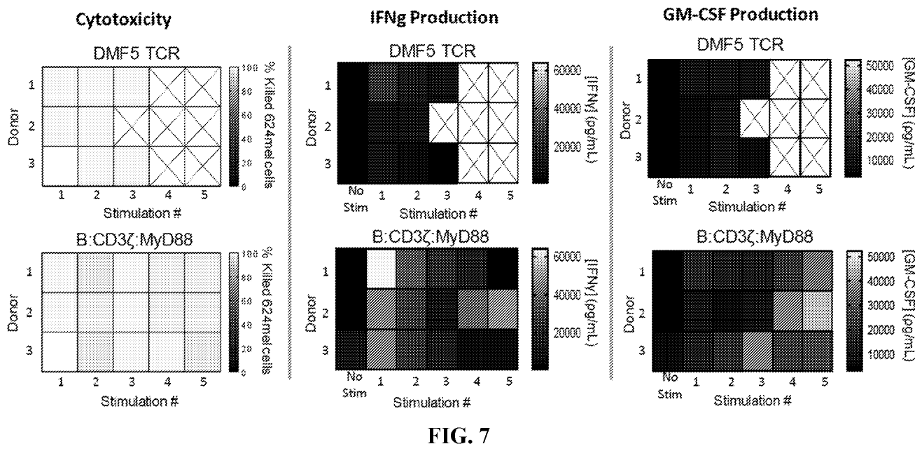

T cells from three donors were engineered with the indicated DMF5 TCRs or and DMF5 TCR in which CD3z:MyD88 was appended to the TCR beta chain. T cells were stimulated with HLA-A2+MART-1+624mel cells (2 tumor: 1 T cell) every 7 days at which point their ability to kill melanoma cells was determined by flow cytometry. Their ability to produce IFNg and GM-CSF was also assessed every 7 days by ELISA. Data are shown in FIG. 7. The TCRB:CD3z:MyD88 T cells maintained their ability to kill cancer cells up to at least 5 rounds of stimulations as compared with control T cells. TCRB:CD3z:MyD88 T cells sustained their ability to produce IFNγ over several rounds of stimulation with cancer cells. Furthermore, TCRB:CD3z:MyD88 conferred upon T cells the ability to not only sustain GM-CSF production but also to improve cytokine production over repeated stimulations.

### Example 7: CD3z:MyD88 Augments Effector Molecules and Promotes the Development of Polyfunctional T Cells

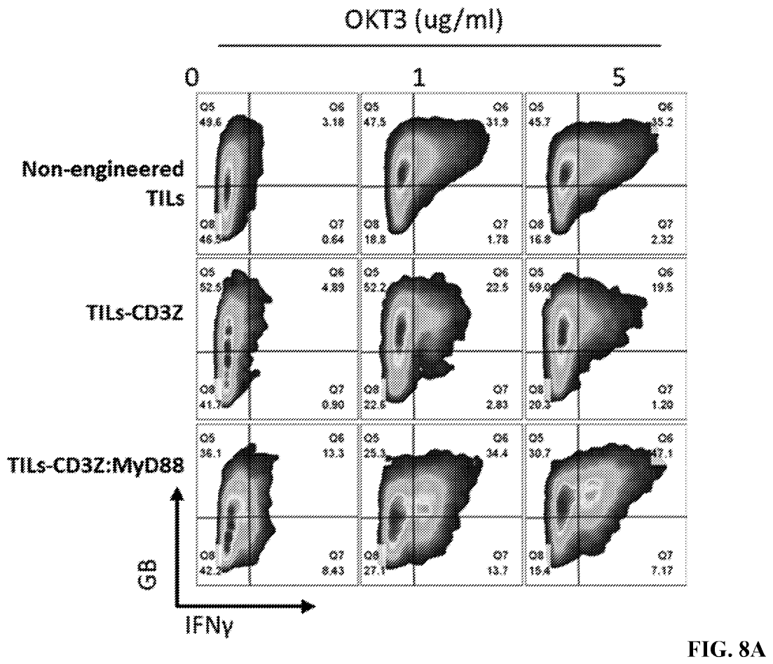

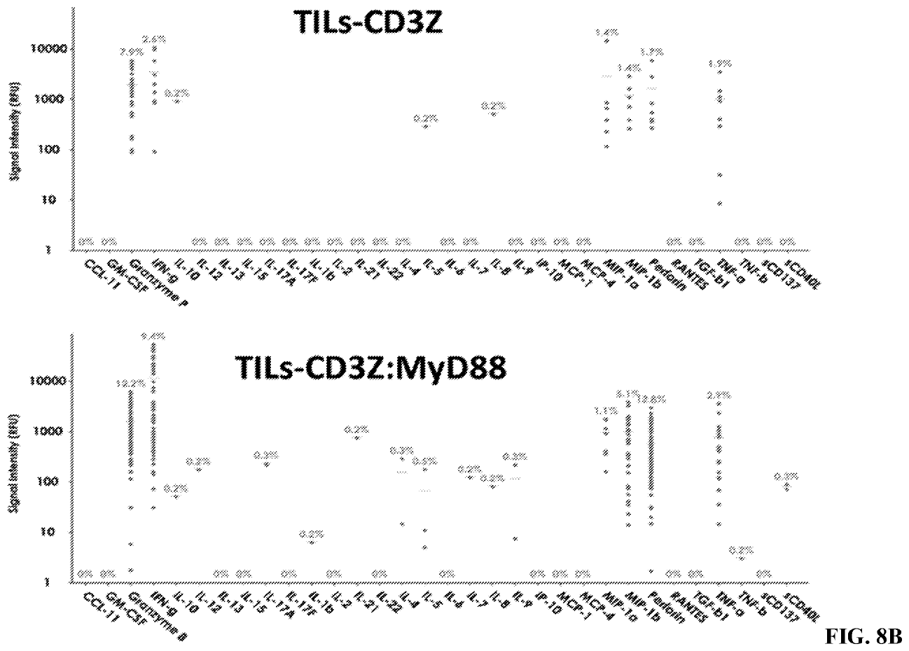

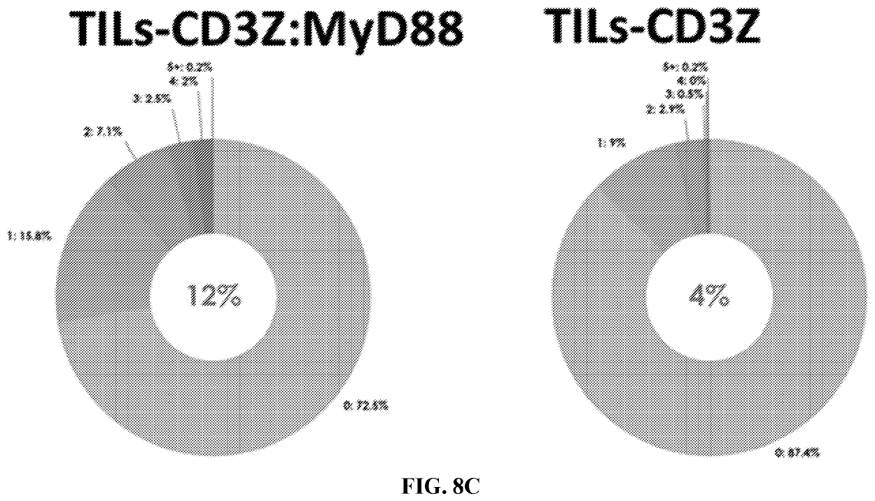

Tumor-infiltrating lymphocytes (TILs) were engineered with either a control CD3z or CD3z:MyD88 or remained non-modified. TILs were then stimulated with plate-bound CD3 Antibody (clone OKT3) for 6 hours, at which time intracellular levels of cytokines were determined by flow cytometry. Results are shown in FIG. 8A and indicate that intracellular interferon gamma (INFγ) increased in a dose-dependent manner. Based on the increased levels of polyfunctional TILs (those that produce more than one cytokine), each individual T cell's ability to co-produce multiple cytokines was assessed using Isoplexus (which measures the indicated cytokines on a per-cell-basis). Results are shown in FIGS. 8B and 8C and indicate that CD3z:MyD88 both augmented the levels of effector molecules and promoted the development of polyfunctional T cells.

### Example 8: CD3z:MyD88 Confers TILs Potent Proliferation Advantages

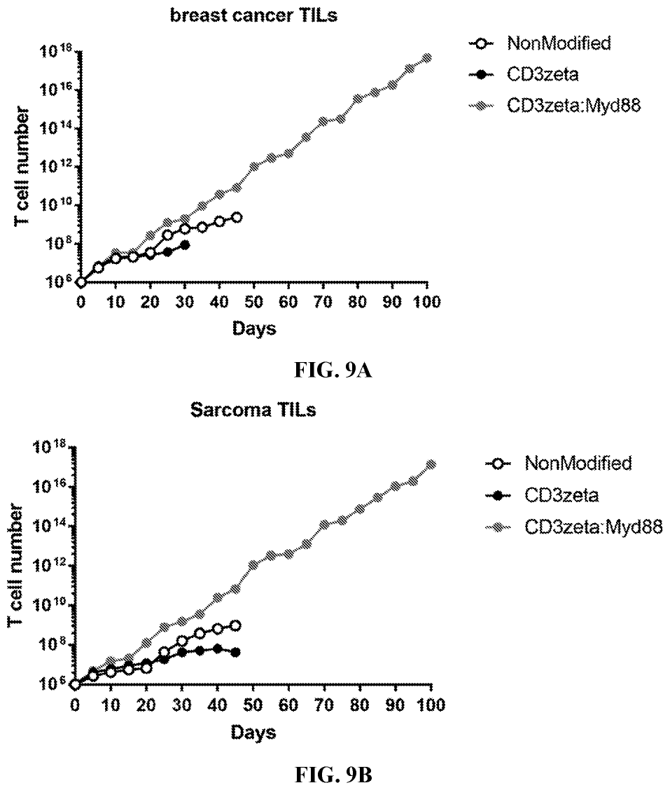

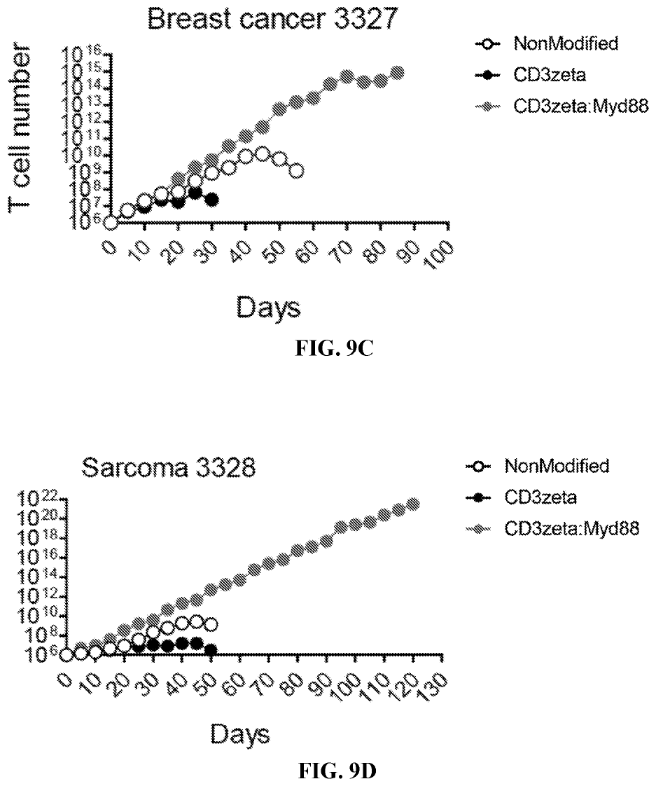

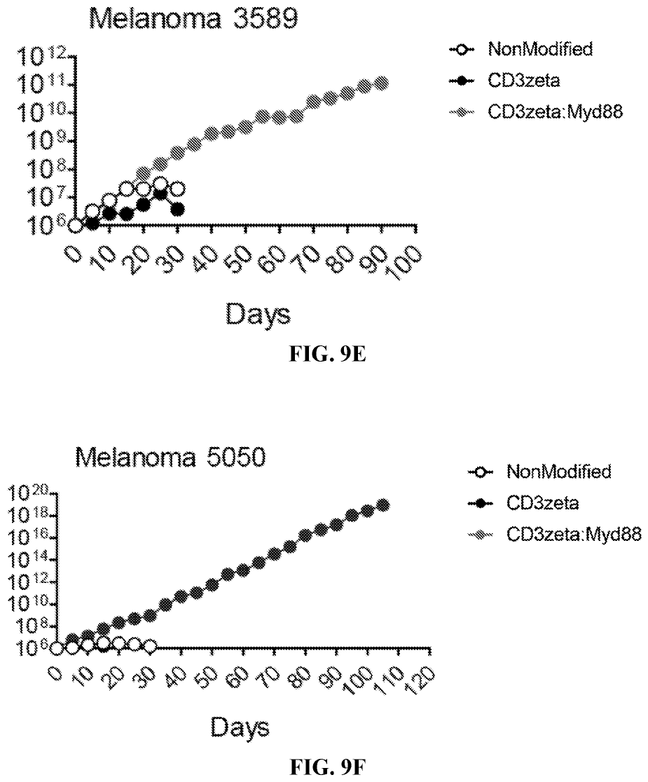

TILs from breast cancer or sarcoma patients were extracted and engineered with the control CD3z or CD3z:MyD88 or remained non-modified. TILs were cultured in the presence of beads coated with anti-CD3 antibody, anti-CD28 antibody, and anti-4-1BB antibody (1:20 Bead:TIL ratio) and IL-2 (500 U/ml) and restimulated every 15 days. Cells were counted every 5-10 days (as indicated in FIGS. 9A and 9F). Results for breast cancer TILs (FIGS. 9A and 9C), sarcoma TILs (FIGS. 9B and 9D) and melanoma TILs (FIGS. 9E and 9F) indicate that TILs transfected with CD3z:MyD88 persisted for up to 100 days (some longer), which was an unexpectedly long survival compared to the control TILs or non-modified TILs, which died within a few weeks. Moreover, the rate of cell expansion was steeper for CD3z:MyD88 TILs compared to CD3z control and untransfected TILs. This data demonstrates a surprising ability to maintain TILs over extended periods of time, which is believed to confer survival advantages of TILs in living hosts as well.

### Example 9: The TCR Diversity and Repertoire is Maintained in CD3z and CD3z:MyD88 Engineered TILs

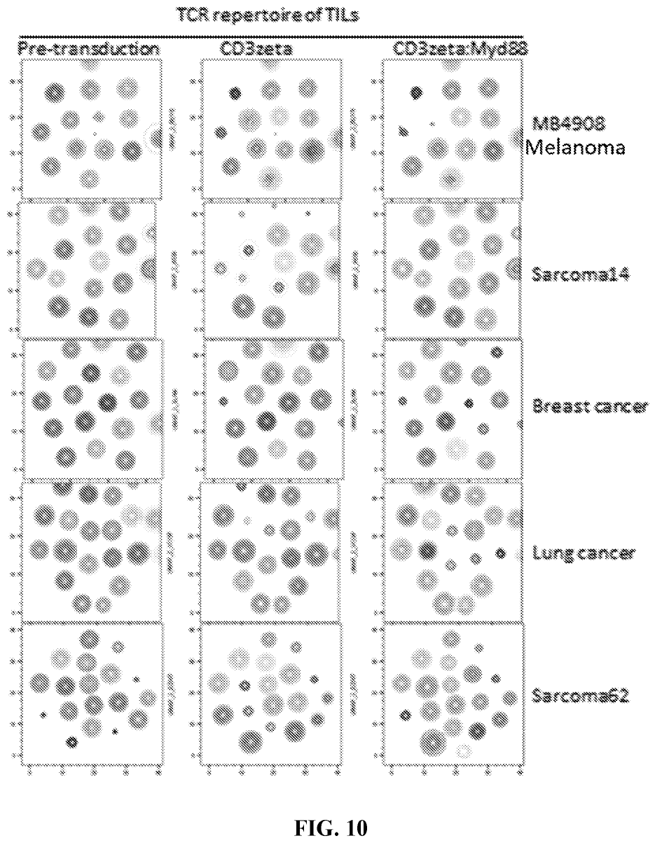

TILs from melanoma, sarcoma, breast cancer, and lung cancer samples were extracted and engineered with the control CD3z or CD3z:MyD88 or remained non-modified (pre-transduction). The diversity of the TCR variable (V) β repertoire was determined in pre-engineered TILs (prior to expansion) or 15 days after expansion (with coated-antibodies beads as described in Example 2) and IL-2 and assessed by flow cytometry. Results are shown in FIG. 10, where each population represents one TCRVβ family member. These data indicate that the TCR diversity and repertoire is maintained in CD3z and CD3z:MyD88 engineered TILs.

### Example 10: CD3z:MyD88 Sustains Polyfunctionality of T Cells Over Prolonged Periods of Time

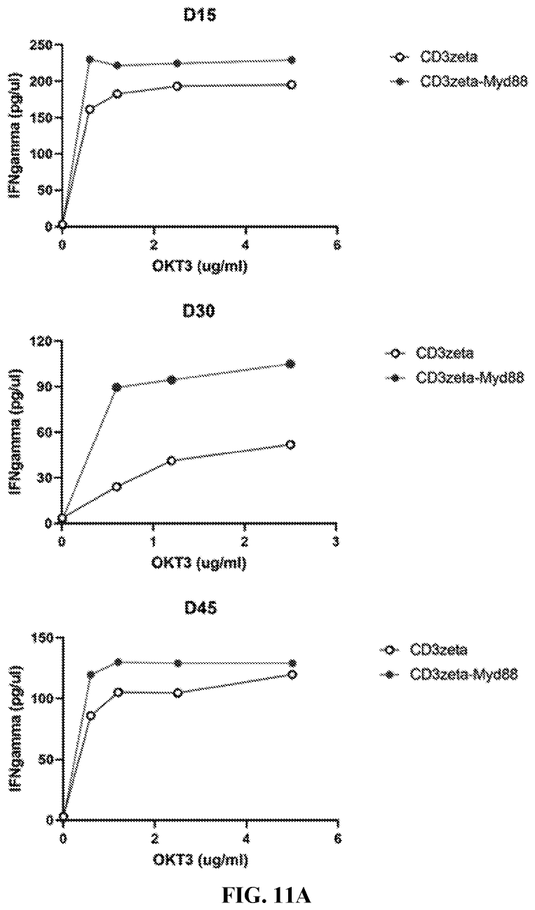

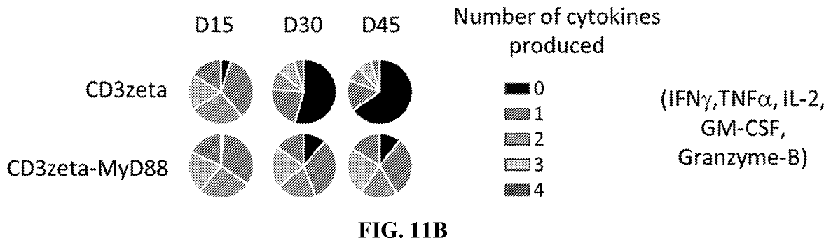

T cells were engineered to express the control CD3z or CD3z:MyD88. T cells were cultured in the presence of beads coated with anti-CD3 antibody, anti-CD28 antibody, and anti-4-1BB antibody (1:20 Bead:TIL ratio) and IL-2 (500 U/ml) and restimulated every 15 days. Just prior to expansion (days 15, 30, and 45) T cells were stimulated with plate-bound anti-CD3 antibody (clone OKT3) for 6 hours at which time intracellular levels of cytokines were determined by flow cytometry. Results of IFNγ expression over time are shown in FIG. 11A and the percentage of T cells capable of co-producing various effector molecules at different time points is shown in FIG. 11B. Most T cells from the control group became exhausted by 30 days. In sharp contrast, CD3z:MyD88 T cells not only retained their ability to produce at least one cytokine, but most also remained polyfunctional.

### Example 11: CD3z:MyD88 Enhances T Cell Activity (Based on IFNg Production)

TILs from the indicated tumor types remained non-engineered or were engineered to express the control CD3z or CD3z:MyD88. T cells were stimulated with the indicated concentrations of plate bound anti-CD3 antibody (clone OKT3) for 6 hrs at which time levels of cytokines were determined by ELISA.

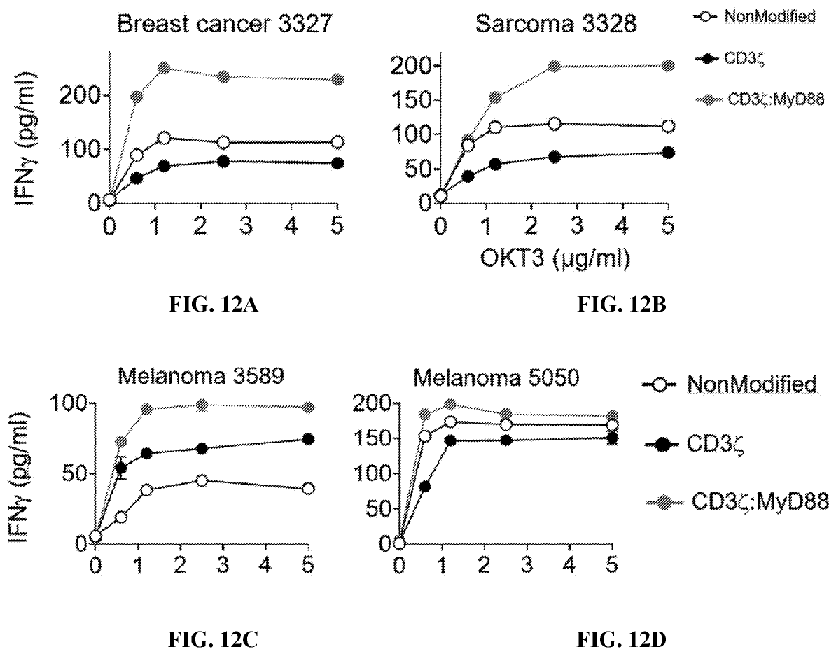

Results are shown in FIGS. 12A-12D for breast cancer (FIG. 12A), sarcoma FIG. 12B) and melanoma (FIGS. 12C and 12D) samples. CD3z:MyD88 amplified the TCR signals across different TCR stimulation intensities resulting in enhanced effector molecule production compared to CD3z and untransfected controls.

### Example 12: CD3z:MyD88 Confers T Cells Proliferation Advantages

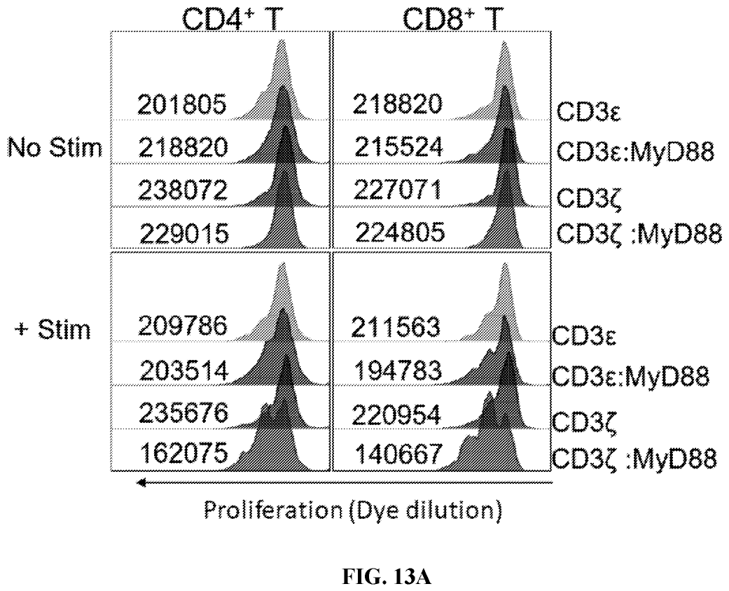

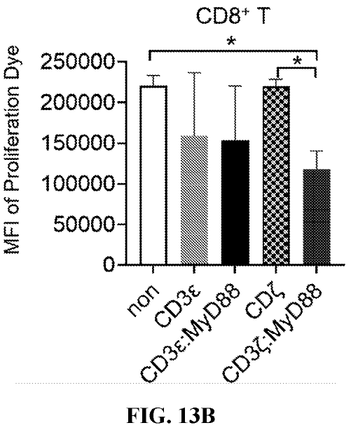

T cells from healthy donors were engineered with the control CD3ε, CD3z,CD3e:MyD88 or CD3z:MyD88. T cells were stained with a proliferation dye and stimulated with anti CD3 (OKT3 clone) and IL 2 (100 U/ml) and proliferation assessed 5 days later. Results are shown in FIGS. 13A and 13B.

CD3z:MyD88 conferred proliferation advantages to both CD4 and CD8 T cells as compared with control groups. However, CD3E:MyD88 did not appear to offer enhanced proliferation over CD3E alone.

### Example 13: CD3z:MyD88 T Cells Offer Potent Antitumor Effects Against Established Melanoma Tumors

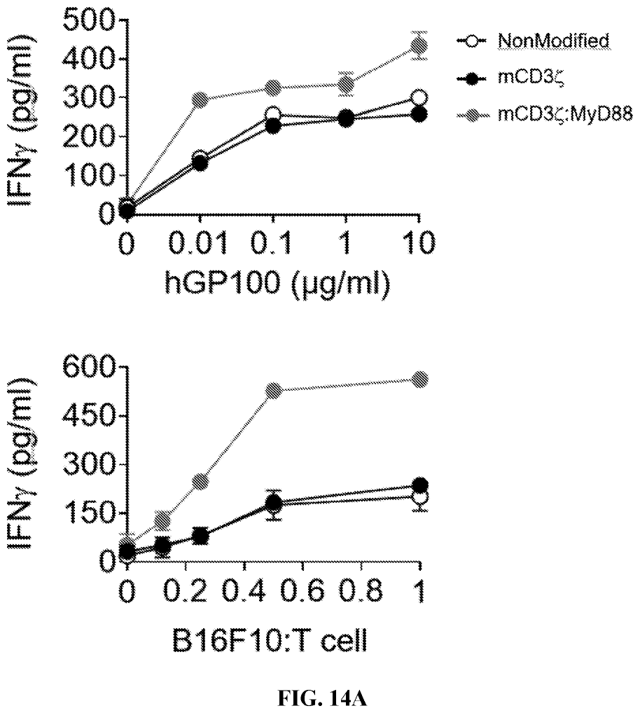

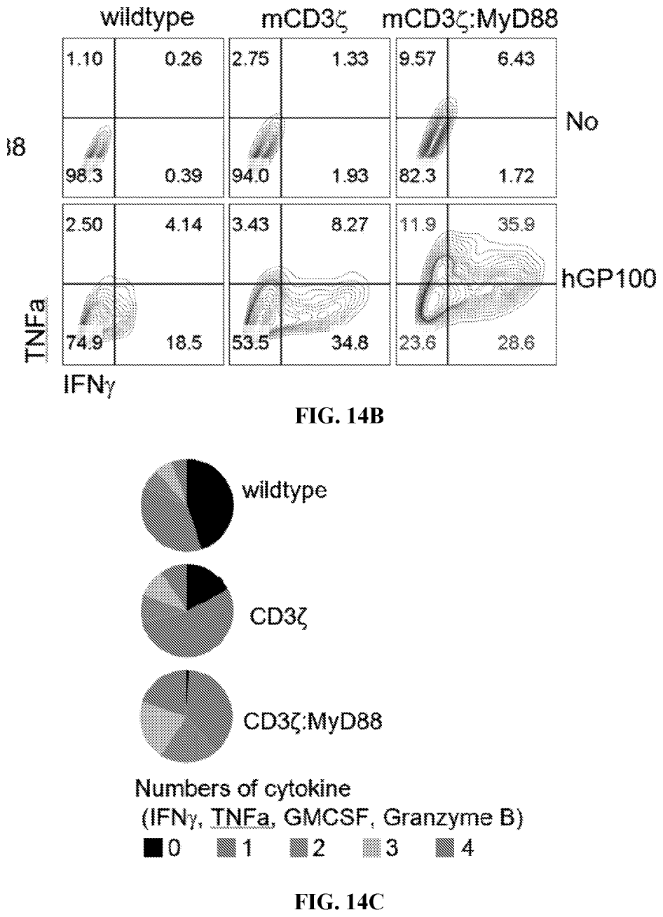

Transgenic mice reactive towards an antigen contained within the gp100 protein remained non-engineered or were engineered to express mCD3z or mCD3z:MyD88. Three days after transduction, T cells were stimulated with B6 splenocytes pulsed varying concentrations of the gp100 antigen or with B16 tumor cells and IFNg production was assessed by ELISA ˜12 hours later. Results are shown in FIG. 14A The production and percentage of TNFa and IFNg were determined by intracellular FACS following 6 hrs stimulation with antigen pulsed splenocytes. Results are shown in FIG. 14B and 14C. These data indicate that CD3z:MyD88 T cells offer potent antitumor effects against established melanoma tumors.

### Example 14: CD3z:MyD88 T Cells Offer Potent Antitumor Effects Against Established Melanoma Tumors

Mice were subcutaneously injected with B16 tumor cells (˜200,000) s.c.. Once tumors were palpable, mice underwent pre-conditioning with 400 cGy radiation and were injected with vehicle control (PBS), or tumor-specific T cells engineered to express the control CD3z or CD3z:MyD88 two days (3E6 cells) and 5 days (4.8E6 cells) after radiation. IL-2 (10KU) was delivered via intraperitoneal injection on the day of the first T cell infusion and two after the first T cell infusion. Tumor growth was monitored using electronic calipers.

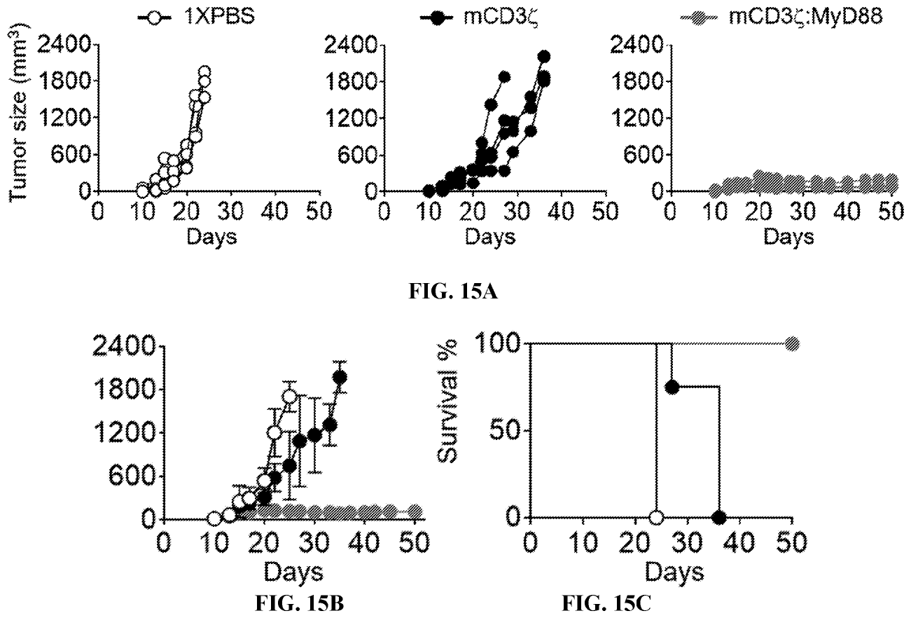

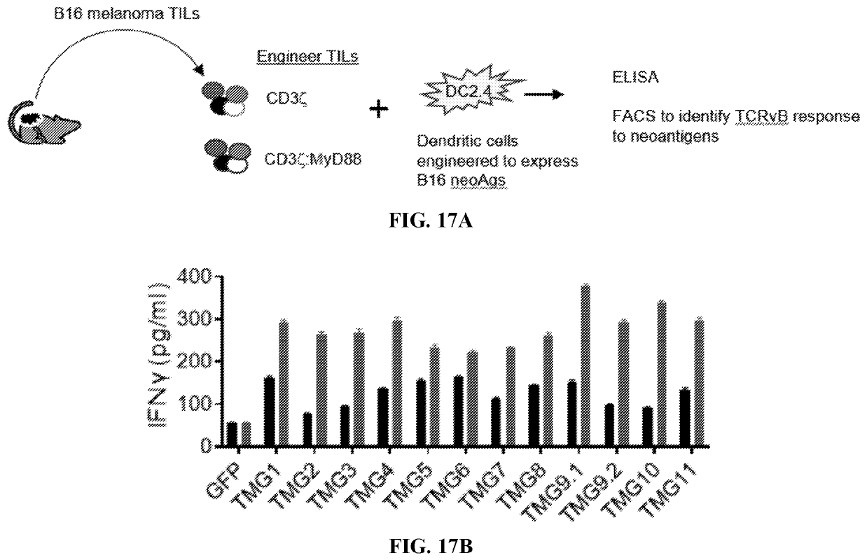

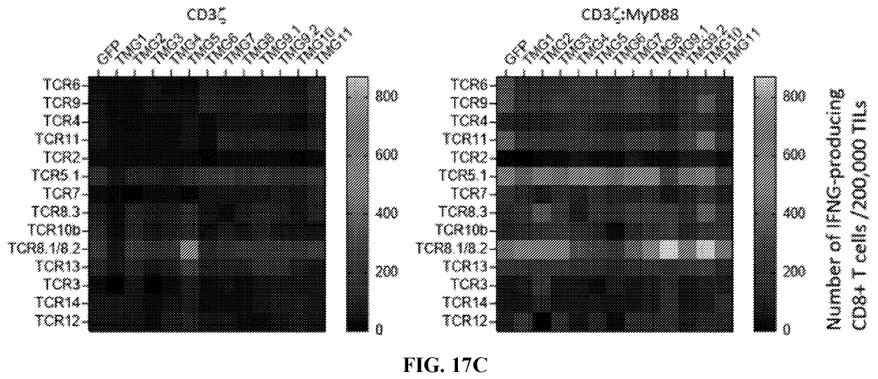

Results for one of 3 representative experiments are shown in FIG. 15A (n=4-5 mice per group). FIG. 15B shows the average results for each group, and FIG. 17C shows percent surviving mice (statistical significance in FIG. 15C was determined by the mixed effect ANOVA model with Geisser Greenhouse correction and Tukey's multiple comparisons test *** p<0.001).

These data show that TILs expressing CD3z:MyD88 were able to prevent tumor growth for the duration of the study (50 days), which is in stark contrast to TILs expressing control CD3z.

Since immunotherapy can be associated with severe immune-related adverse events, including cachexia (a hallmark of this in mice and humans), weight loss was monitored in all treatment groups.

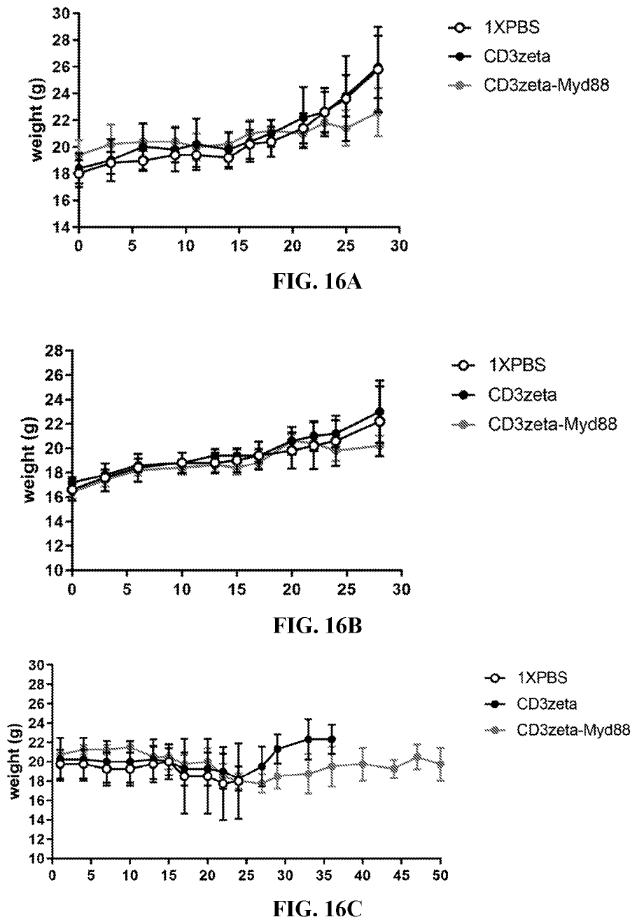

Data for three different experiments is presented in FIGS. 16A-16C. These results show that, in three different experiments, the rate of weight recovery in tumor-bearing mice following treatment were comparable between groups.

### Example 15: CD3z:MyD88 Awakens Neoantigen Reactive TILs

CD8 TILs were extracted from B16 F10 tumors and engineered to express CD3z or CD3z:MyD88. Dendritic cells were engineered to express plasmids containing tandem mini genes (TMG) expressing 10 neoantigen each. DCs were co cultured with engineered TILs for ˜24 hours later. FIG. 17A shows a schematic of the experimental set up. The levels of IFNg were determined by ELISA and the number of IFNg producing CD8 T cells as well the usage of TCRvB gamily usage was determined by flow cytometry. Results are shown in FIGS. 17B and 17C. These data indicate that CD3z:MyD88 can awaken neoantigen reactive TILs.

What is claimed is:

1. A cell comprising
(a) a modified T cell receptor (TCR) comprising an α chain, a β chain, a CD3ζ intracellular domain and a MyD88 polypeptide comprising an amino acid sequence that is at least 90% identical to the sequence set forth in SEQ ID NO: 1 or 2, wherein the MyD88 polypeptide is fused to the CD3ζ subunit, or
(b) a fusion protein comprising an amino acid sequence that is at least 90% identical to the amino acid sequence set forth in SEQ ID NO: 3 or 9.

2. The cell of claim 1, wherein the modified TCR comprises an amino acid sequence that is at least 90% identical to the amino acid sequence set forth in SEQ ID NO: 11.

3. The cell of claim 1 or 2 wherein the cell is an immune cell.

4. The cell of any one of claims 1-3, wherein the immune cell is a T cell or a tumor infiltrating lymphocyte (TIL).

5. A pharmaceutical composition comprising the cell of any one of claims 1-4 and a pharmaceutically acceptable carrier.

6. A method of treating cancer in a subject in need thereof, the method comprising administering to the subject the cell of any one of claims 1-4 or the pharmaceutical composition of claim 5.

7. The method of claim 6, wherein the cancer is breast cancer, sarcoma, melanoma, or lung cancer.

8. A method of improving a therapeutic cell, the method comprising modifying a receptor of the therapeutic cell to fuse a MyD88 domain to a CD3ζ domain present in the receptor of the therapeutic cell.

9. The method of claim 8, wherein the MyD88 domain comprises an amino acid sequence that is at least 90% identical to the sequence set forth in SEQ ID NO: 1 or 2.

10. A method of improving a therapeutic cell, the method comprising introducing into the cell a fusion protein comprising a CD3ζ intracellular domain and a MyD88 DD.

11. The method of claim 10, wherein the fusion protein comprises an amino acid sequence that is at least 90% identical to the amino acid sequence set forth in SEQ ID NO: 3 or 9.

12. The method of any one of claims 8-11, wherein the cell is an immune cell.

13. The method of any one of claims 8-11, wherein the cell is derived from a patient.

14. The method of any one of claims 8-13, wherein the method results in an increase in expansion of the therapeutic cell in vivo of at least 50% compared to the therapeutic cell prior to improvement.

15. The method of any one of claims 8-13, wherein the method results in an increase in cytokine expression of the therapeutic cell in vivo of at least 50% compared to the therapeutic cell prior to improvement.

16. The method of any one of claims 8-13, wherein the method results in a decrease in exhaustion of the therapeutic cell in vivo of at least 20% compared to the therapeutic cell prior to improvement.
